(19) United States
(12) Patent Application Publication
(10) Pub. No.: US 2026/0183472 A1
(43) Pub. Date: Jul. 2, 2026

(54) CONTROLLING MEDICAL FLUID INFUSION FOR PATIENTS

(71) Applicant: Astral Med Solutions, Inc., Las Vegas, NV (US)

(72) Inventor: Deepali Mittal, Los Altos, CA (US)

(21) Appl. No.: 19/002,275

(22) Filed: Dec. 26, 2024

(51) Int. Cl. A61M 5/14; A61L 2/10; A61M 5/142; A61M 5/155; A61M 5/165; A61M 5/168; G16H 20/17; G16H 40/63

(52) U.S. Cl. CPC A61M 5/1408; A61L 2/10; A61M 5/14228; A61M 5/155; A61M 5/165; A61M 5/16813; G16H 20/17; G16H 40/63; A61L 2202/11; A61M 2005/14208; A61M 2202/064; A61M 2205/3334; A61M 2205/3344; A61M 2205/6009; A61M 2205/6072; A61M 2205/75

(57) ABSTRACT

The present disclosure discloses system for controlling medical fluid infusion for patients. The system includes storage module for storing plurality of medication containers, plurality of pressurizing pumps, one or more carrier conduits, patient conduit connected to one or more carrier conduits, plurality of pressure valves arranged in association with one or more carrier conduits, or patient conduit, memory, and processor. The processor is configured to receive medication data associated with medication fluid stored in each of plurality of medication containers. The processor is configured to receive patient data associated with patient. The processor is configured to generate set of control parameters associated with one or more pressurizing pumps of plurality of pressurizing pumps, and one or more pressure valves of plurality of pressure valves. The processor is configured to control flow of treatment fluid based on set of control parameters to deliver treatment fluid to patient.

### TECHNICAL FIELD

The present disclosure relates to intravenous infusion systems. More specifically, the present disclosure relates to systems and methods for controlling medical fluid infusions for patients.

### BACKGROUND

Intravenous (IV) medication and fluid infusion devices are widely used in healthcare for the administration of medications, fluids, and nutrients directly into the bloodstream of a patient. Therefore, safe and accurate infusion of medications is critical for medical treatments. Examples of medical treatments may include but are not limited to intravenous (IV) therapy, chemotherapy, dialysis, and anesthesia delivery. Existing IV infusion devices are often large and cumbersome machines that take a considerable amount of time to set up. Further, such infusion devices often rely on manual processes. For example, during operation, the infusion devices require manual input from healthcare professionals (such as nurses, doctors, pharmacists, and the like) to ensure correct patient identification, correct medication, and dosage, correct prescription by the physician, and assign correct personnel to administer the medication. This may result in increased time for administering medication and fluid, thereby reducing operational efficiency. Further, such a manual intervention may lead to challenges such as inaccurate dosages, contamination risks, or suboptimal treatment outcomes. Additionally, the IV devices provide relatively easy access to controlled substances, such as narcotics, for healthcare professionals. This raises concerns about potential misuse, theft, or diversion of such controlled substances.

Generally, the IV medications are administered under sterile conditions to prevent iatrogenic infections. In such a scenario, any remaining IV medication is typically discarded after each dose. This leads to significant medication waste, thereby contributing to environmental pollution. Further, such medications may be expensive, therefore such a disposal of the medications may add to operation costs, thereby increasing treatment costs. For example, issues such as medication incompatibilities between two or more medications that are administered together may arise, thereby leading to blockages in IV lines or ports, rendering them ineffective or unusable. This may result in delays in treatment and additional interventions to correct these issues.

Therefore, there is a need for improved devices and methods for providing infusion treatments that address these inefficiencies while maintaining patient safety, reducing waste, and minimizing environmental impact.

### SUMMARY

In an aspect, a system for controlling medical fluid infusion for patients is provided. The system includes a storage module for storing a plurality of medication containers. Each of the plurality of medication containers stores a medication fluid. The system includes a plurality of pressurizing pumps. Each of the plurality of pressurizing pumps is arranged in connection with a corresponding medication container of the plurality of medication containers, and each of the plurality of pressurizing pumps is configurable to apply a pressure to the corresponding medication container of the plurality of medication containers. The system includes one or more carrier conduits. Each of the one or more carrier conduits is connected to at least one of the plurality of medication containers. The system includes a patient conduit connected to the one or more carrier conduits. The patient conduit is configurable to connect to a patient. The system includes a plurality of pressure valves arranged in association with at least one of the one or more carrier conduits, or the patient conduit. The system includes a memory configured to store computer-executable instructions and one or more processors coupled to the storage module, the plurality of pressurizing pumps, and the plurality of pressure valves. The one or more processors are configured to execute the computer-executable instructions to receive medication data associated with the medication fluid stored in each of the plurality of medication containers and receive patient data associated with the patient. The patient data includes prescription data. The processor generates dosage data for an infusion of a treatment fluid to the patient based on the medication data and the patient data. The dosage data indicates a concentration associated with one or more medication fluids stored in the one or more medication containers of the plurality of medication containers for forming the treatment fluid. The processor generates a set of control parameters associated with one or more pressurizing pumps of the plurality of pressurizing pumps, and one or more pressure valves of the plurality of pressure valves. The set of control parameters is generated based on the medication data and the dosage data, and the one or more pressurizing pumps are associated with the one or more medication containers. The processor controls a flow of the treatment fluid based on the set of control parameters to deliver the treatment fluid to the patient.

In an embodiment, at least one of the plurality of medication containers corresponds to a multi-chamber container. The multi-chamber container includes a first chamber configured to store a first medication entity. The first medication entity corresponds to at least one of a powdered state, a solid state, or a semi-solid state. The multi-chamber container includes a second chamber configured to store a second medication entity. The second medication entity corresponds to a liquid state. The multi-chamber container includes a mixing mechanism associated with the multi-chamber container. The mixing mechanism is coupled to the one or more processors.

In an embodiment, the one or more processors are further configured to identify the multi-chamber container from the plurality of medication containers based on the dosage data. The first medication entity and the second medication entity of the multi-chamber container are combined to form the treatment fluid. The processor generates the set of control parameters based on the identification. The processor controls the mixing mechanism to combine the first medication entity and the second medication entity to form the treatment fluid based on at least a part of the set of control parameters.

In an embodiment, the second medication entity is a pressurized fluid, and the pressurized fluid corresponds to one of: a gas, a liquid, a Newtonian fluid, a non-Newtonian fluid to increase a pressure in the multi-chamber container.

In an embodiment, the plurality of pressurizing pumps corresponds to compressed gas pumps. Each of the plurality of pressurizing pumps is configured to apply the pressure for the corresponding medication container of the plurality of medication containers using a compressed gas.

In an embodiment, the plurality of pressurizing pumps corresponds to peristaltic pumps. Each of the plurality of pressurizing pumps is configured to apply the pressure for the corresponding medication container of the plurality of medication containers using a compressible flexible tubing.

In an embodiment, the plurality of pressurizing pumps corresponds to hydraulic pumps. Each of the plurality of pressurizing pumps is configured to apply the pressure for the corresponding medication container of the plurality of medication containers using a liquid medium.

In an embodiment, the treatment fluid includes a medication fluid stored within a medication container of the plurality of medication containers or a combination of a set of medication fluids stored within a set of medication containers of the plurality of medication containers.

In an embodiment, the one or more processors are further configured to identify the one or more medication containers from the plurality of medication containers based on the dosage data. The medication fluid from each of the one or more medication containers is combined to form the treatment fluid. The processor identifies the one or more pressure valves from the plurality of pressure valves. The one or more pressure valves are associated with at least one of a connection between the identified one or more medication containers and at least one carrier conduit of the one or more carrier conduits, the at least one carrier conduit, or the patient conduit. The processor generates the set of control parameters for controlling each of the one or more pressurizing pumps and the one or more pressure valves. The one or more pressurizing pumps are associated with the identified one or more medication containers, and the set of control parameters indicates a pressure value and a flow rate.

In an embodiment, the one or more processors are further configured to determine pressure data based on the dosage data. The pressure data includes a first pressure value associated with the one or more pressurizing pumps, a second pressure value associated with the connection between each of the one or more medication containers and the at least one carrier conduit, a third pressure value associated with the at least one carrier conduit, and a fourth pressure value associated with the patient conduit. The processor generates the set of control parameters for controlling each of the one or more pressurizing pumps and the one or more pressure valves, based on the pressure data.

In an embodiment, the second pressure value is lesser than the first pressure value, the third pressure value is lesser than the second pressure value, and the fourth pressure value is lesser than the third pressure value, and the control of each of the one or more pressurizing pumps and the one or more pressure valves is based on the corresponding set of control parameters, such that the pressure data prevents backflow and backdiffusion of the treatment fluid and contamination of any one of the at least one carrier conduit, the patient conduit, or the plurality of medication containers.

In an embodiment, the patient conduit is configurable to connect to a subsequent patient after the delivery of the treatment fluid to the patient. The patient conduit connected to the patient is discarded after each use and a new sterile patient conduit is affixed to the system prior to a subsequent patient use. The one or more processors are configured to cause to deliver the treatment fluid or new treatment fluid to the patient.

In an embodiment, a first label is positioned on each of the plurality of medication containers. The system further includes a scanner. The scanner is configured to read the first label of each of the plurality of medication containers, and the one or more processors are configured to retrieve the medication data associated with each of the plurality of medication containers based on the reading.

In an embodiment, the system includes a plurality of filter modules. Each of the plurality of filter modules is arranged in connection with at least one of the one or more carrier conduits, or the patient conduit, each of the plurality of filter modules includes at least one microbe filter to filter one or more microbe particles, and each of the plurality of filter modules includes at least one blood cell filter to filter blood-particulate matter.

In an embodiment, the processor is further configured to receive filter data associated with each of the plurality of filter modules. The filter data includes at least one of pressure change data, usage data, expiration data, anomaly data, or flow data. The processor determines performance data associated with each of the plurality of filter modules. The performance data indicates a current filtering capacity of each of the plurality of filter modules. The processor generates notifications data based on the performance data. The notification data is generated based on a determination of the current filtering capacity to be less than a capacity threshold.

In an embodiment, each of the at least one microbe filter and the at least one blood cell filter are selected based on a pore size.

In an embodiment, a second label is positioned on each filter module of the plurality of filter modules. The system further includes a scanner. The scanner is configured to read the second label of each of the plurality of filter modules. The one or more processors are configured to retrieve the filter data associated with each of the plurality of filter modules based on the reading.

In an embodiment, the system includes a sterilization module arranged in connection with each of the plurality of filter modules. The sterilization module is configured to periodically sterilize each of the plurality of filter modules.

In an embodiment, the sterilization module is configured to sterilize each of the one or more filter modules using at least one of an ultraviolet (UV) light, or chemical agent.

In an embodiment, the one or more processors are further configured to apply an artificial intelligence (AI) model to the medication data and the patient data. The processor generates the dosage data based on the application of the AI model to the medication data and the patient data. The dosage data indicates one or more delivery parameters to deliver the treatment fluid to the patient.

In an embodiment, the one or more processors are further configured to obtain real-time patient vitals data associated with the patient, apply the artificial intelligence (AI) model to the real-time patient vitals data, and generate the dosage data based on the application of the AI model to the real-time patient vitals data. The patient vitals data includes at least one of: height, weight, sex, age, temperature, blood pressure, heart rate, saturation, central venous saturation, heart rhythm, erythrocyte sedimentation rate (SED) Line monitor information, electroencephalogram (EEG) information, nerve stimulator information, or neuro-monitoring information.

In an embodiment, the system further includes a dispensing pump to deliver the treatment fluid from the patient conduit to the patient. The one or more processors are further configured to obtain flow data associated with a flow of the treatment fluid from the patient conduit to the patient. The flow data is obtained from the dispensing pump. The system is configured to apply the AI model to the flow data. The one or more processors are further configured to update the dosage data based on the application of the AI model to the flow data.

In an embodiment, the one or more delivery parameters indicate at least one of the one or more medication fluids for forming the treatment fluid, a mixing ratio for forming the treatment fluid, a mixing method for the treatment fluid, a time period for the delivery of the treatment fluid to the patient, a concentration of the treatment fluid, or a flow rate of the treatment fluid.

In an embodiment, the one or more processors are further configured to identify an anomaly associated with at least one of the one or more medication containers based on the medication data, the flow data, and the AI model. The processor generates action data based on the identified anomaly. The action data includes at least one of one or more resolution parameters for resolving the anomaly, or notification data for a notification associated with the anomaly.

In an embodiment, the one or more processors are further configured to receive the biometric data. The biometric data is associated with a healthcare professional. The processor validates rights of the healthcare professional to use the system based on the biometric data. These rights may include rights to create, modify or follow the prescription data. The processor generates the dosage data for the infusion of the treatment fluid to the patient based on the validation.

In an embodiment, the plurality of medication containers includes one or more sets of medication containers, such that one or more medication containers in a set of medication containers are connected in a series configuration with one of the one or more carrier conduits, and each set of the one or more sets of medication containers is connected in a parallel configuration with the one or more carrier conduits.

In another aspect, a method for controlling medical fluid infusion for patients is provided. The method includes storing a plurality of medication containers in a storage module. Each of the plurality of medication containers stores a medication fluid. The method includes connecting each of one or more carrier conduits to at least one of the plurality of medication containers. The method includes connecting a patient conduit to the one or more carrier conduits. The patient conduit is configurable to connect to a patient. The method includes arranging a plurality of pressure valves in association with at least one of the one or more carrier conduits, or the patient conduit. The method includes receiving medication data associated with the medication fluid stored in each of the plurality of medication containers. The method includes receiving patient data associated with the patient, the patient data includes prescription data. The method includes generating dosage data for an infusion of a treatment fluid to the patient based on the medication data and the patient data. The dosage data indicates a concentration associated with one or more medication fluids stored in the one or more medication containers of the plurality of medication containers for forming the treatment fluid. The method includes generating a set of control parameters associated with one or more pressurizing pumps of a plurality of pressurizing pumps, and one or more pressure valves of the plurality of pressure valves. The set of control parameters is generated based on the medication data and the patient data, and the one or more pressurizing pumps are associated with the one or more medication containers. The method includes controlling a flow of the treatment fluid based on the set of control parameters to deliver the treatment fluid to the patient.

In an embodiment, the method includes identifying a multi-chamber container from the plurality of medication containers based on the dosage data. The multi-chamber container includes a first chamber configured to store a first medication entity, a second chamber configured to store a second medication entity, and a mixing mechanism associated with the multi-chamber container, and the first medication entity and the second medication entity of the multi-chamber container are combined to form the treatment fluid. The method includes generating the set of control parameters based on the identification; and controlling a mixing mechanism to combine the first medication entity and the second medication entity to form the treatment fluid based on at least a part of the set of control parameters.

In an embodiment, the method includes identifying the one or more medication containers from the plurality of medication containers based on the dosage data. The medication fluid from each of the one or more medication containers is combined to form the treatment fluid. The method includes identifying the one or more pressure valves from the plurality of pressure valves. The one or more pressure valves are associated with at least one of a connection between the identified one or more medication containers and at least one carrier conduit of the one or more carrier conduits, or at least one carrier conduit of the one or more carrier conduits and the patient conduit. The method includes generating the set of control parameters for controlling each of the one or more pressurizing pumps and the one or more pressure valves. The one or more pressurizing pumps are associated with the identified one or more medication containers, and the set of control parameters indicates a pressure value and a flow rate.

In an embodiment, the method includes determining pressure data based on the dosage data. The pressure data includes a first pressure value associated with the one or more pressurizing pumps, a second pressure value associated with the connection between each of the one or more medication containers and the at least one carrier conduit, a third pressure value associated with the at least one carrier conduit, and a fourth pressure value associated with the patient conduit. The method includes generating the set of control parameters for controlling each of the one or more pressurizing pumps and the one or more pressure valves, based on the pressure data. The second pressure value is lesser than the first pressure value, the third pressure value is lesser than the second pressure value, and the fourth pressure value is lesser than the third pressure value.

In an embodiment, the method includes applying an artificial intelligence (AI) model to the medication data, the patient data and the prescription data. The method includes generating the dosage data based on the application of the AI model to the medication data and the patient data. The dosage data indicates one or more delivery parameters to deliver the treatment fluid to the patient.

In an embodiment, the method includes obtaining real-time patient vitals data associated with the patient, applying the artificial intelligence (AI) model to the real-time patient vitals data, and generating the dosage data based on the application of the AI model to the real-time patient vitals data. The patient vitals data includes at least one of: height, weight, sex, age, temperature, blood pressure, heart rate, saturation, central venous saturation, heart rhythm, erythrocyte sedimentation rate (SED) Line monitor information, electroencephalogram (EEG) information, nerve stimulator information, or neuro-monitoring information.

In an embodiment, the method includes receiving filter data associated with each of a plurality of filter modules. Each of the plurality of filter modules is arranged in connection with at least one of the one or more carrier conduits, or the patient conduit. Each of the plurality of filter modules includes at least one microbe filter to filter one or more microbe particles and at least one blood cell filter to filter blood-particulate matter, and the filter data includes at least one of pressure change data, usage data, expiration data, anomaly data, or flow data. The method includes determining performance data associated with each of the plurality of filter modules. The performance data indicates a current filtering capacity associated with each of the plurality of filter modules associated with each of the plurality of filter modules. The method includes generating notifications data based on the performance data. The notification data is generated based on a determination of the current filtering capacity to be less than a capacity threshold.

In yet another aspect, a computer programmable product comprising a non-transitory computer-readable medium having stored thereon computer-executable instructions, which when executed by one or more processors, cause the one or more processors to conduct operations, the operations including receiving medication data associated with a medication fluid stored in each of a plurality of medication containers. The plurality of medication containers is stored in a storage module, and each of the plurality of medication containers stores a medication fluid. The operations include receiving patient data associated with a patient, the patient data includes prescription data and real-time patient vitals data. The operations include applying an artificial intelligence (AI) model to the medication data, the patient data and the prescription data. The operations include generating dosage data for an infusion of a treatment fluid to the patient based on the application of the AI model to the medication data and the patient data. The dosage data indicates a concentration associated with one or more medication fluids stored in the one or more medication containers of the plurality of medication containers for forming the treatment fluid. The operation includes generating a set of control parameters associated with one or more pressurizing pumps of a plurality of pressurizing pumps, and one or more pressure valves of a plurality of pressure valves. The set of control parameters is generated based on the medication data and the dosage data, and the one or more pressurizing pumps are associated with the one or more medication containers. The operations include controlling a flow of the treatment fluid based on the set of control parameters to deliver the treatment fluid to the patient.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

Having thus described example embodiments of the invention in general terms, reference will now be made to the accompanying drawings, which are not necessarily drawn to scale, and wherein:

FIG. 1A illustrates an exemplary block diagram of a system for controlling medical fluid infusion for patients, in accordance with an embodiment of the present disclosure;

FIG. 1B illustrates another exemplary block diagram of the system for controlling medical fluid infusion for patients, in accordance with an embodiment of the present disclosure;

FIG. 2 is a schematic diagram of a storage module, in accordance with an embodiment of the present disclosure;

FIG. 3 illustrates a schematic diagram of the system for controlling medical fluid infusion for patients, in accordance with an embodiment of the disclosure;

FIG. 4 illustrates a schematic diagram of conduits of the system for controlling medical fluid infusion for patients, in accordance with an embodiment of the disclosure;

FIG. 5 illustrates another schematic diagram of conduits of the system for controlling medical fluid infusion for patients, in accordance with an embodiment of the disclosure;

FIG. 6A illustrates a schematic diagram of a multi-chamber container storing medication fluid, in accordance with an embodiment of the disclosure;

FIG. 6B illustrates another schematic diagram of a multi-chamber container storing medication fluid, in accordance with an embodiment of the disclosure;

FIG. 7 illustrates a block diagram of exemplary operations for controlling medical fluid infusion for patients, in accordance with an embodiment of the disclosure;

FIG. 8 illustrates a flowchart of a method for generating notification data, in accordance with an embodiment of the disclosure;

FIG. 9 illustrates a block diagram to generate dosage data, in accordance with an embodiment of the disclosure;

FIG. 10 illustrates a flowchart of a method for generating action data, in accordance with an embodiment of the disclosure;

FIG. 11 illustrates a flowchart of a method for generating dosage data, in accordance with an embodiment of the disclosure;

FIG. 12 illustrates an exemplary block diagram of the system for controlling medical fluid infusion for patients, in accordance with an embodiment of the present disclosure; and

FIG. 13 illustrates a flowchart of an exemplary method for controlling medical fluid infusion for patients, in accordance with an embodiment of the disclosure.

### DETAILED DESCRIPTION OF DRAWINGS

In the following description, for purposes of explanation, numerous specific details are set forth in order to provide a thorough understanding of the present disclosure. It will be apparent, however, to one skilled in the art that the present disclosure may be practiced without these specific details. In other instances, systems and methods are shown in block diagram form only in order to avoid obscuring the present disclosure.

Some embodiments of the present disclosure will now be described more fully hereinafter with reference to the accompanying drawings, in which some, but not all, embodiments of the disclosure are shown. Indeed, various embodiments of the disclosure may be embodied in many different forms and should not be construed as limited to the embodiments set forth herein; rather, these embodiments are provided so that this disclosure will satisfy applicable legal requirements. Like reference numerals refer to like elements throughout. Also, reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced items. Moreover, various features are described which may be exhibited by some embodiments and not by others. Similarly, various requirements are described which may be requirements for some embodiments but not for other embodiments.

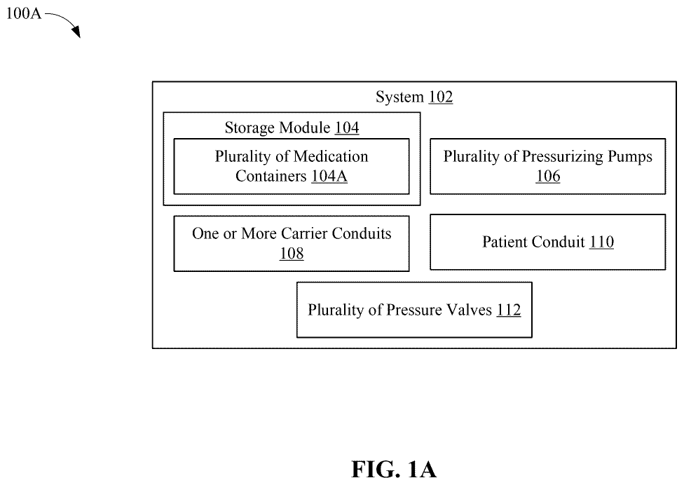

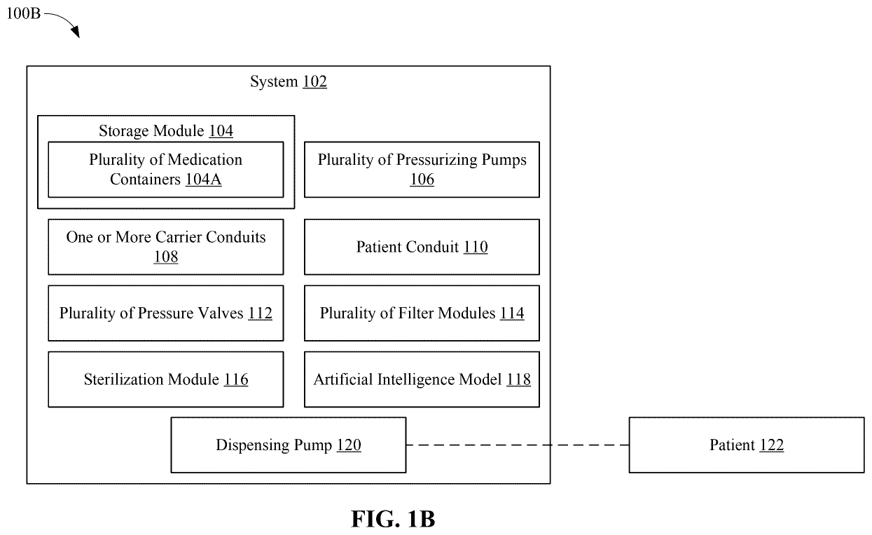

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect. Turning now to FIG. 1-FIG. 13, a brief description concerning the various components of the present disclosure will now be briefly discussed. Reference will be made to the figures showing various embodiments of the apparatus for controlling medical fluid infusion for patients.

Intravenous (IV) medication refers to fluids or medicines that may be administered directly into the bloodstream of a patient. Existing IV infusion devices are often large, cumbersome, and require a significant amount of time to set up. Such a delay may be partly due to the necessity of verifying various parameters, including correct patient identification, medication order verification, medication type and dosage verification, verification of the prescribing healthcare provider, and confirmation that qualified personnel are administering the medication. Such a verification of the parameters is essential to prevent medication errors. However, such verification of the parameters may contribute to increased time in administering the medication and inefficiencies in medical workflows. For example, in high-pressure environments like emergency rooms or intensive care units, such a time-consuming verification process may lead to a bottleneck in providing rapid and effective care.

Further, healthcare providers (including nurses, doctors, and pharmacists) may have access to controlled substances, particularly narcotics, thereby making it a critical concern with IV infusion devices. This may lead to a risk of misuse, theft, or diversion of the medication and fluid substances. Moreover, the existing devices typically mandate the disposal of any remaining medication after it has been administered to a patient, thereby ensuring sterility to prevent iatrogenic infections caused by medical treatments or treatments. However, such disposal of medication results in significant waste, particularly when expensive medications are involved, thereby contributing to higher costs of treatments and increasing the environmental burden from the disposal of these substances. Further, the disposal of unused medications, particularly in liquid form, may cause environmental harm. Additionally, hazardous chemicals may infiltrate water systems, posing a risk to ecosystems and public health. Therefore, reducing medication waste is not only an economic concern but also an environmental imperative. There may be a need to develop a system that minimizes leftover medication and protects both financial and natural resources.

The proposed system seeks to reduce medication errors, such as incorrect medication, wrong dosage, or patient misidentification. These errors have been linked to severe adverse outcomes and even fatalities. The root causes often include similar-looking medication vials, leading to confusion during preparation, inconsistent labeling practices, and improper dosing when administering medications into syringes or mixing them with carrier fluids. Therefore, the proposed system enhances patient safety and improves clinical outcomes thereby reducing such risks. Additionally, the proposed system is designed to reduce the waste associated with leftover medications. Such an improvement prevents costly medications from being discarded unnecessarily and lessens the negative environmental impact of pharmaceutical waste. Therefore, the proposed system aligns healthcare practices with broader environmental goals, thereby promoting better resource management and sustainability.

Additionally, the proposed system streamlines the process of setting up IV medication systems, thereby saving valuable time for healthcare professionals. For example, automated checks for patient identity, medication type, dosage, and prescribing information may potentially replace manual verifications, reducing human error and accelerating the administration process. This could be particularly beneficial in critical care settings, where every minute counts. Further, the proposed system mitigates medication incompatibility issues between two or more medications that may be administered together, which may lead to blockages in IV lines. Such blockages may indicate medications ineffective or make IV ports and lines unusable, thereby compromising patient care. By addressing these compatibility issues, the proposed system may improve the reliability of IV medication delivery and reduce the need for intervention due to line blockages.

Therefore, the proposed system ensures that the IV medication and fluid are administered safely and securely, thereby minimizing the potential for abuse. Further, the system enhances patient safety, reduces waste, and protects the environment. The proposed system leverages the integration of artificial intelligence into IV medication administration, thereby fostering a safer, more efficient, and more environmentally responsible healthcare system.

FIG. 1A illustrates an exemplary block diagram 100A of a system 102 for controlling medical fluid infusion for patients, in accordance with an embodiment of the present disclosure. The system 102 includes a storage module 104, a plurality of pressurizing pumps 106, one or more carrier conduits 108, a patient conduit 110, and a plurality of pressure valves 112. The storage module 104 includes a plurality of medication containers 104A.

The system 102 corresponds to an intravenous (IV) medication and fluid infusion device. The system 102 allows rapid administration of the IV medication fluid directly into the bloodstream of a patient. Further, the system 102 may be designed to manage and administer one or more medication fluids to the patient in an automated, precise, and efficient manner so as to provide accurate dosage of one or multiple medications to the patient. For example, the system 102 may ensure the safe and controlled delivery of the medication fluid. The medication fluid refers to a liquid substance or combination of substances used in the medical treatment of the patient. The medication fluid may include, but is not limited to, medications, saline solutions, electrolytes, blood, nutrients, or other therapeutic substances intended for infusion.

The storage module 104 refers to a cabinet, housing, or a designated unit designed to securely store the plurality of medication containers 104A. The storage module 104 may be customizable and/or selectable. For example, the storage module may include but is not limited to shelves or compartments to provide organized storage and easy access to the stored plurality of medication containers 104A. In an example, the storage module 104 may further include, but is not limited to, a temperature control mechanism, or a locking mechanism to protect the stored plurality of medication containers 104A. For example, the storage module 104 may provide refrigeration and/or heating of some or all the medications and/or fluids. The storage module 104 securely stores the plurality of medication containers 104A and keeps the one or more medication fluids sterile until needed. For example, the storage module 104 houses the medication fluid within the plurality of medication containers 104A. For example, the plurality of medication containers 104A may be designed using different materials, such as but not limited to, glass, plastic, polymer, or other materials compatible with the medication fluid. Further, the plurality of medication containers 104A serves as a primary reservoir from which the medication fluid is drawn for administering to the patient. The plurality of medication containers 104A stores the medication fluid in various forms, such as a liquid form, a powdered form, a semi-solid form, or a combination thereof. For example, the plurality of medication containers 104A may store the medication fluid like, but not limited to saline, antibiotics, or specialized medications, ready to be dispensed as required for the treatment. For example, the medication fluid may be drawn, combined, and delivered to the patient using the plurality of medication containers 104A. In another example, the plurality of medication containers 104A may correspond to a multi-chamber container to store and mix medication components before administration to the patient. For example, the multi-chamber container may include a first chamber and a second chamber. In such an example, the first chamber may store a powdered medication, or a solid medication and the second chamber may store a fluid medication configured to solubilize the powdered or solid medication prior to the delivery to the patient.

The plurality of medication containers 104A may be connected to the plurality of pressurizing pumps 106 and the one or more carrier conduits 108. The plurality of pressurizing pumps 106 may generate and maintain a pressure for the movement of the medication fluid, thereby accurately delivering the medication fluid to the patient through the one or more conduits 108. For example, the plurality of pressurizing pumps 106 may correspond to a mechanical device or an electronic device configured to pressurize a corresponding medication container of the plurality of medication containers 104A. For example, the plurality of pressurizing pumps 106 utilizes pressurized gases such as carbon dioxide, nitrogen, and/or inert gases to generate the pressure. In another example, the plurality of pressurizing pumps 106 utilizes a proprietary gas/solvent mixture to control a pressure if carbon dioxide is used to apply the pressure. Further, the plurality of pressurizing pumps 106 may be arranged with a corresponding medication container of the plurality of medication containers 104A and apply the pressure thereof. Further, the plurality of pressurizing pumps 106 may regulate the flow rate and pressure to ensure precise mixing and delivery of the medication fluid. The plurality of pressurizing pumps 106 may be configured to adapt to varying fluid viscosities and flow demands based on a user input. For example, the plurality of pressurizing pumps 106 may include but is not limited to a compressed gas mechanism, a peristaltic pump, a hydraulic pump, or other pressurizing pumps. For example, each of the plurality of pressurizing pumps 106 apply pressure to a first medication container of the plurality of medication containers 104A and cause the medication fluid from a corresponding medication container of the plurality of medication containers 104A to flow and pushes the medication fluid from the corresponding medication container of the plurality of medication containers 104A through the one or more carrier conduits 108 towards the patient conduit 110.

The one or more carrier conduits 108 and the patient conduit 110 refer to a tubular pathway or a channel utilized to transport the medication fluid from the one or more medication containers 104A to the patient. The one or more carrier conduits 108 and the patient conduit 110 may incorporate connections, valves, or filters. In an example, the one or more carrier conduits 108 may include one or more valves of the plurality of pressure valves 112 for controlled delivery and to ensure the seamless mixing of the medication fluids to form the treatment fluid. Further, the patient conduit 110 may also include one or more valves of the plurality of pressure valves 112 for controlled delivery of the treatment fluid to the patient. In an example, the patient conduit 110 interfaces with the patient to administer the treatment fluid at the prescribed rate and pressure while maintaining sterility and preventing contamination, backflow and/or backdiffusion.

The plurality of pressure valves 112 may be configured to regulate a flow of the medication fluid by modulating pressure within the one or more conduits 108 or the patient conduit 110. The plurality of pressure valves 112 may be strategically arranged in association with the one or more conduits 108 or the patient conduit 110. The plurality of pressure valves 112 facilitates controlling flow rates and pressure levels of dispensing of the medication fluid, thereby ensuring precise dosage delivery and preventing backflow, backdiffusion or cross-contamination within the system 102. For example, a pressure valve may be arranged at a dispensing end of the one or more medication containers. In another example, a pressure valve may be arranged at a dispensing end of the one or more carrier conduits 108 or the patient conduit 110.

In an example, the plurality of pressure valves 112 may correspond to specialized valves that can achieve both functions: preventing backflow of fluid and preventing backdiffusion of small molecules. Such pressure valves are typically used in systems where both fluid control and selective molecular exclusion are critical. For example, standard check valves may prevent backflow of fluid by allowing flow in only one direction. If such check valves are equipped with high-quality seals (e.g., elastomeric or PTFE), they can also limit diffusion of small molecules under certain condition. Such check valves may be used in liquid chromatography, chemical processing, and medical devices. In an example, membrane check valves incorporated with a semi-permeable membrane acts as both a barrier to small molecules and a one-way flow mechanism. The membrane material and pore size determine effectiveness in blocking small molecules, is ideal for biological or chemical systems requiring selective diffusion control. In another example, non-return valves (NRV) include coatings or internal linings designed to resist permeation of gases and small molecules. The NRV combines traditional check valve mechanisms with a molecular barrier and are commonly used in high-purity chemical systems or systems where small molecule diffusion must be minimized (e.g., oxygen-sensitive environments). In yet another example, a multi-function valves with active control may correspond to advanced valves that may combine check valve functionality with an integrated molecular filtration system (such as activated carbon or zeolites) to adsorb or block small molecules while controlling fluid direction. Such valves may be used in environmental sampling, fuel systems, and sensitive analytical equipment. Therefore, while choosing an appropriate pressure value, it is important to ensure that the pressure valve is rated for a required operating pressure and is compatible with the medication fluids being used in the system 102. Further, ensure the pressure valve or membrane is effective against the specific molecule size that may be targeted.

In operation, the storage module 104 may include the plurality of medication containers 104A that may store the medication fluid. The medication fluid may correspond to a plurality of prescription or non-prescription IV medications and/or IV fluids including but not limited to controlled and/or non-controlled medications and fluids. The medication fluid refers to a liquid substance or combination of substances used in the medical treatment of the patients. The medication fluids may include medications, chemotherapeutics, saline solutions, electrolytes, nutrients, or other therapeutic substances intended for infusion. The medication fluid is stored in each of the plurality of medication containers 104A and combined to form a treatment fluid. Further, the storage module 104 may include the plurality of medication containers 104A. The plurality of medication containers 104A may correspond to cartridges or other types of glass, plastic, or polymer containers, or containers from any material compatible with the medication fluid, which contain a specific medication type, medication concentration, and/or medication quantity.

The system 102 may determine the quantity of medication present in the plurality of medication containers 104A using sensors. In an example, the sensors may monitor the flow of the medication fluid in real-time, detecting issues such as blockages, leaks, or abnormal pressure in the one or more carrier conduits 108 or the patient conduit 110. This would provide healthcare providers with immediate feedback and prevent potential complications such as extravasation (fluid leaking into surrounding tissue) or dosage errors. Further, the system 102 may determine a presence of bubbles of air and/or other gases in the plurality of medication containers 104A, the one or more carrier conduits 108, pressure valves 112, and/or filter modules 114. For example, the plurality of medication containers 104A may be pressurized by gas, hydraulic, or mechanical means using the plurality of pressurizing pumps 106. The plurality of medication container 104A may include special linings to ensure safe and stable storage of the medication fluid. For example, the plurality of medication container 104A may include a septum or septa to separate different components of the container, such as a lyophilized medication from a saline or sterile water solution or an aqueous medication from the plurality of pressurizing pumps 106. The storage module 104 may include a mechanism that allows for the selection and/or movement of each of the plurality of medication containers 104A. Such a movement may allow easier access for each of the plurality of pressurizing pumps to the medication containers within the storage module 104. Further, the storage module 104 may allow for loading of each of the plurality of medication containers 104A, maintenance of pressure in each of the plurality of medication containers 104A, pressurizing of each of the plurality of medication containers 104A, measurement of pressure in the each of the plurality of medication containers 104A, and depressurization of each of the plurality of medication containers 104A.

The system 102 is configured to receive the medication data associated with the medication fluid stored in each of the plurality of medication containers 104A. The medication data for a medication fluid in a medication container may include, but is not limited to, composition and properties associated with the medication fluid. Further, the system 102 is configured to receive patient data associated with the patient. The patient data may refer to information associated with the health of the patient. For example, the patient data may include but is not limited to personal details, medical history, and prescription data. The personal details may include, for example, but are not limited to name, age, gender, contact details, insurance details, lifestyle data, body weight, height, blood type, medication allergies, medication reactions and biometric data. The prescription data may include, but is not limited to, medical prescriptions and treatment requirements for the patient. The medical history may include, but is not limited to, historical medical treatments over a period of time, historical diagnoses, treatments, lab results, historical appointments, and billing records.

Thereafter, system 102 is configured to generate dosage data for an infusion of the treatment fluid to the patient based on the medication data and the patient data. The dosage data indicates a concentration associated with one or more medication fluids stored in the one or more medication containers of the plurality of medication containers 104A for forming the treatment fluid. The treatment fluid refers to one medication fluid or a combination of medication fluids prepared for infusion into the patient. The treatment fluid includes one or more medication fluids in specific concentrations and ratios. Further, the treatment fluid includes a medication fluid stored within a medication container of the plurality of medication containers or a combination of a set of medication fluids stored within one or more of medication containers of the plurality of medication containers 104A. For example, the treatment fluid is formed by combining medication fluids from one or more containers of the medication containers 104A based on the medication data and the prescription data. Further, the treatment fluid is delivered to the patient through the one or more carrier conduits 108 and the patient conduit 110 after being regulated by the pressurizing pumps 106 and the pressure valves 112. In an example, one or more treatment fluids or medication fluids are to be administered to the patient. Further, the one or more treatment fluids or medication fluids may not be compatible to be infused together, the system 102 may include one or more patient conduits connected to the patient to administer the one or more treatment fluids or medication fluids. Thereafter, the one or more patient conduits (e.g. a physical tubing attached to the patient) may be discarded after each use.

The dosage data refers to the specific information regarding the quantity, timing, and duration of medication or treatment fluid to be administered to the patient. The dosage data is typically determined based on the patient's medical condition, weight, age, or prescription guidelines. Further, the system 102 may leverage the use of the medication data and the patient data to calculate dosage data tailored to the patient. For example, the dosage data for a chemotherapy patient might specify a slow, continuous infusion of 200 ml over four hours, and the system 102 adjusts flow rates accordingly.

Further, the system 102 is configured to generate a set of control parameters associated with one or more pressurizing pumps of the plurality of pressurizing pumps 106, and one or more pressure valves of the plurality of pressure valves 112. The set of control parameters may be generated based on the medication data and the dosage data. Further, the system 102 may generate the set of control parameters tailored to the patient. The system 102 may leverage the use of the generated set of control parameters to deliver a precise amount of medication to the patient, thereby ensuring accuracy and reducing the risk of under-dosing or overdosing. The set of control parameters may include flow rates, pressure levels, sterilization cycles, and medication compatibility data. The set of control parameters enables the system 102 to function within safe limits. The system 102 may dynamically adjust the set of control parameters based on any changes in the medication data, the patient data and/or the dosage data using the one or more pressurizing pumps 106 associated with the one or more medication containers 104A. For example, the set of control parameters may include a maximum flow rate (such as but not limited to 15 ml/min) for the flow of the medication fluid that may not be exceeded for a specific infusion line, thereby ensuring patient safety. In an example, the set of control parameters includes user-specific values that regulate the operation flow of the treatment fluid. For example, a healthcare professional (such as, a nurse or doctor) may interact with the system 102. Further, the healthcare professional may input commands, monitor operations, and adjust settings through a user interface. For instance, the healthcare professional may modify infusion rates or pause operations based on patient responses. In another example, the set of control parameters includes the dynamic values that regulate the operation flow of the treatment fluid.

Thereafter, the system 102 is configured to control a flow of the treatment fluid based on the set of control parameters to deliver the treatment fluid to the patient. The flow of the treatment fluid may be controlled to provide an accurate dosage of the treatment fluid to the patient. In an example, a dispensing pump may be connected to the system 102. Further, the patient conduit 110 may be connected to the patient through the dispensing pump. Moreover, the system 102 may control the flow rate of the treatment fluid through the patient conduit 110 and the dispensing pump to provide the treatment fluid to the patient. In an example, the system may control the operation of the plurality of pressurizing valves 112 and the plurality of pressurizing pumps 106 to control the flow of the treatment fluid to the patient.

FIG. 1B illustrates another exemplary block diagram 100B of the system 102 for controlling medical fluid infusion for the patients, in accordance with an embodiment of the present disclosure. FIG. 1B is explained in conjunction with elements from FIG. 1A. The system 102 includes the storage module 104, the plurality of pressurizing pumps 106, the one or more carrier conduits 108, the patient conduit 110, and the plurality of pressure valves 112. The storage module 104 includes the plurality of medication containers 104A. The system 102 may further include a plurality of filter modules 114, a sterilization module 116, an artificial intelligence (AI) module 118, and a dispensing pump 120.

The plurality of filter modules 114 are specialized devices used to ensure the safety and efficacy of the system 102 by purifying the medication fluids or treatment fluids before their administration to a patient 122. The patient 122 may correspond to a user to which the medication fluid is to be administered. The plurality of filter modules 114 may be configured to remove microorganisms, particulate matter, or specific cellular components that may compromise the sterility or safety of the fluids. The system 102 may employ the plurality of filter modules 114 to prevent infections or complications by eliminating harmful particulates or microorganisms from the fluid pathway. For example, during an intravenous infusion, the plurality of filter module 114 ensures that the saline, blood products or medication fluid delivered to the patient is free of particulates or bacterial contaminants. For example, the plurality of filter modules 114 may include but is not limited to a microbial filter, a particulate filter, or a blood cell filter.

The microbial filter may be designed to remove or block microorganisms such as but not limited to bacteria, viruses, or fungi, from the saline, blood products or the medication fluid. For example, the microbial filter may be of round or cylindrical shape to fit into a syringe or IV line. Further, the microbial filter may be compact in size for small-scale use. Additionally, a pore size of the microbial filter may be 0.2 microns or smaller to remove bacteria and viruses. In an example, the microbial filter may be made of a material such as but not limited to polycarbonate material, nylon, or ceramic to withstand sterilization and pressure.

The blood cell filter may be designed to remove unwanted blood components such as but not limited to white blood cells, platelets, cellular debris from a blood product. This may ensure a safe transfusion of blood. For example, the blood cell filter may be of flat or cylindrical shape to fit into a blood transfusion set. Further, the blood cell filter may be compact to allow easy integration into the IV line. Additionally, a pore size of the blood cell filter may be fine to remove blood cells without affecting plasma or smaller components. In an example, the blood cell filter may be made of a biocompatible material, such as but not limited to polyester, or polyethylene to prevent immune reactions.

The sterilization module 116 may be configured to disinfect and maintain sterility of the medication fluid pathways, the one or more carrier conduits 108, and other system components that encounter the medication fluids. The sterilization module 116 may be arranged in connection with each of the plurality of filter modules 114. The sterilization module 116 may be configured to periodically sterilize each of the plurality of filter modules 114. Further, the sterilization module 116 ensures that the system 102 remains free from contamination, especially when switching between patients or after prolonged use. Further, the sterilization module 116 maintains a sterile environment, reducing the risk of infections or cross-contamination. Additionally, the sterilization module 116 may be configured to sterilize each of the one or more filter modules using an ultraviolet (UV) light and/or chemical agent. Examples of the chemical agents may include, but are not limited to ethylene oxide, hydrogen peroxide, or chlorine compounds. In an example, after completing an infusion for one patient, the sterilization module may perform a cleaning cycle, using steam or chemical disinfectants, to prepare the system 102 for the next use.

The AI model 118 may be a computational network or a system of artificial neurons, arranged in a plurality of layers, as nodes. The plurality of layers of the AI model 118 may include an input layer, one or more hidden layers, and an output layer. Each layer of the plurality of layers may include one or more nodes (or artificial neurons). Outputs of all nodes in the input layer may be coupled to at least one node of the hidden layer(s). Similarly, inputs of each hidden layer may be coupled to outputs of at least one node in other layers of the AI model 118. Outputs of each hidden layer may be coupled to inputs of at least one node in other layers of the AI model 118. Node(s) in the final layer may receive inputs from at least one hidden layer to output a result (such as the AI image data). The number of layers and the number of nodes in each layer may be determined from hyper-parameters of the AI model 118. Such hyper-parameters may be set before or while the training of the AI model 118 on a training dataset.

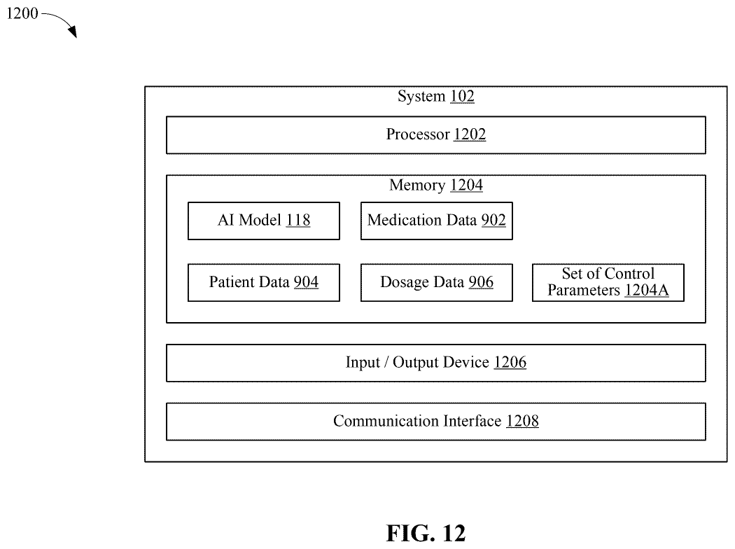

The AI model 118 may include electronic data, such as, for example, a software program, code of the software program, libraries, applications, scripts, or other logic or instructions for execution by a processing device, such as a processor set. The AI model 118 may include code and routines configured to enable a computing device, such as the system 102 to perform one or more operations. Additionally, or alternatively, the AI model 118 may be implemented using hardware including a processor, a microprocessor (e.g., to perform or control performance of one or more operations), a field-programmable gate array (FPGA), or an application-specific integrated circuit (ASIC). Alternatively, in some embodiments, the AI model 118 may be implemented using a combination of hardware and software. Accordingly, in some embodiments, the AI model 118 may be stored in the memory 1204, see FIG. 12. Examples of the AI model 118 may include, but are not limited to, a deep neural network (DNN), a convolutional neural network (CNN), a CNN-recurrent neural network (CNN-RNN), an artificial neural network (ANN), a fully connected neural network, and/or a combination of such networks.

The dispensing pump 120 may be configured to control the delivery of the treatment fluid to the patient 122. The dispensing pump 120 may regulate the flow rate of the treatment fluid and ensures that the prescribed dosages are administered accurately to the patient 122. For example, the dispensing pump 120 may ensure precise control over the treatment fluid, as defined by the dosage data and patient-specific requirements. Further, the dispensing pump 120 works in tandem with other modules, such as the pressure valves 112 and the pressurizing pumps 106, to maintain the required flow rate. For example, the dispensing pump 120 may deliver a steady flow of 10 ml/min of an antibiotic solution during an infusion therapy session.

In operation, the system 102 may be configured to receive filter data associated with each of the plurality of filter modules 114. The filter data may include, but is not limited to, pressure change data, usage data, expiration data, anomaly data, or flow data. The pressure change data refers to variations in pressure across the plurality of filter modules 114 during operation. This helps to determine when the filter needs cleaning or replacement. The usage data may track the operation history of the filter, including a duration of use and a volume of fluid processed. The expiration data indicates a date beyond which the filter may no longer perform effectively or remain sterile. The anomaly data may indicate unusual patterns or deviations in the performance of the filter such as but not limited to pressure change, flow irregularities, or leaks. The flow data may include information associated with movement and measurement of the medication fluid through the filter.

The system 102 may determine performance data associated with each of the plurality of filter modules 114. The performance data indicates a current filtering capacity of each of the plurality of filter modules 114. For example, the current filtering capacity indicates the ability of the filter to process and remove contaminants from the medication fluid. Further, the system 102 generates notification data based on the performance data. The notification data may include a message or an alert to indicate a status associated with the performance of the filter, or when maintenance is required. For example, the notification data is generated based on a determination of the current filtering capacity to be less than a capacity threshold.

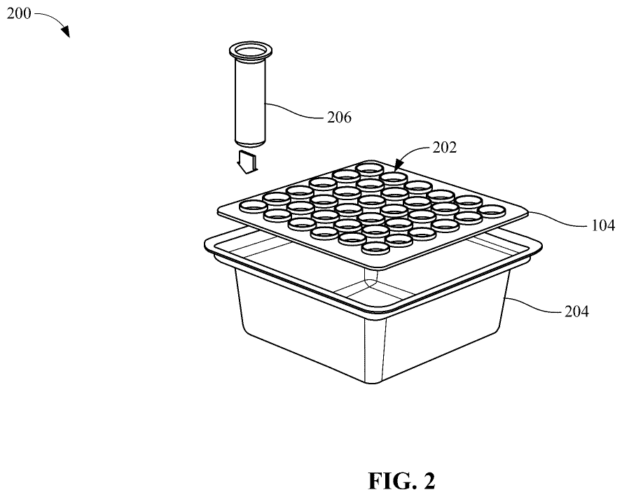

The functions or operations executed by the system 102 are described in detail, for example, in conjunction with FIG. 2-FIG. 13.

FIG. 2 is a schematic diagram of a storage module 104, in accordance with an embodiment of the present disclosure. FIG. 2 is explained in conjunction with elements from FIG. 1A and FIG. 1B. The diagram 200 includes the storage module 104 corresponding to a multi-compartment module for storing a plurality of medication containers 104A. Each of an individual compartment 202 of the multi-compartment module may hold each medication container of the plurality of medication containers 104A. Further, each medication container 206 of the plurality of medication containers 104A stores a medication fluid. The storage module 104 may correspond to an organizing tray that fits into a case (such as the container compartment 204). The system 102 is configured to utilize the received patient data, specifically, the prescription data of the patient 122 to select the medication fluid to be provided to the patient 122. The system 102 may include a dispensing pump 120 that allows for the movement of the medication fluid towards the patient 122 from the medication containers using the patient conduit 110. Such a movement may allow easier access for the plurality of pressurizing pumps 106 to the plurality of medication containers 104A to allow the movement of the medication fluid from a medication container to the one or more carrier conduits 108 to perform mixing of the one or more medication fluid.

The storage module 104 includes a physical library of medications, which includes the actual medication containers 206 and their associated storage mechanisms such as the individual compartment 202 within the container compartment 204. The storage module 104 is a structured, organized repository of intravenous (IV) medications and fluids designed to house and maintain a physical stock of the medication fluids. Further, the storage module 104 may contain both prescription and non-prescription medications, including controlled substances (e.g., opioids, anesthetics) and non-controlled fluids (e.g., saline, dextrose solutions). Additionally, the storage module 104 may include transfer means that may be responsible for storage, pressurization, and preparation of the medication fluids for delivery. The transfer means may include the plurality of pressurizing pumps 106, the one or more carrier conduits 108, the patient conduit 110, the plurality of pressure valves 112, and the dispensing pump 120.

The storage module 104 organizes medication containers 104A systematically to maintain accessibility and proper environmental conditions for medication preservation. Some medication fluids may need to be stored separately and mixed before administration. In such an instant, each medication container 206 of the plurality of medication containers 104A in the storage module 104 may include multi-compartment designs, with a septum for separating different components. For example, each medication container 206 of the plurality of medication containers 104A house medication fluids in glass, plastic, polymer containers, or other materials compatible with the medication fluids. The medication containers 104A are designed to hold specific medication types, concentrations, and quantities. Some containers such as the medication container 206 of the plurality of medication containers 104A may incorporate special linings to prevent chemical degradation and ensure long-term stability, which is especially important for sensitive medications like chemotherapy agents. For example, each medication container 206 of the plurality of medication containers 104A may have glass or polymer linings in the inner part of the medication containers 206 to prevent contamination and maintain medication integrity. For example, a glass-lined cartridge could store sensitive medications like insulin to enhance stability.

For example, a plastic cartridge may be lined with glass, to combine the durability and flexibility of plastic with the chemical resistance and inert nature of glass. For example, syringes and cartridges used in pharmaceuticals have a plastic exterior for strength and lightweight handling, while the interior is lined with glass to prevent chemical reactions between the contents and the container. For example, specialized glass coatings or inserts may be applied inside plastic containers to provide a barrier that prevents chemical interaction, maintains purity, and ensures that the contents do not absorb any unwanted compounds from the plastic. In certain industries, glass-lined plastic is used to store and transport reactive or sensitive substances, offering both resistance to breakage (due to the presence of plastic) and high chemical compatibility (due to the presence of glass). There are several manufacturers known for producing glass-lined plastic vials, syringes, or cartridges, often for specialized applications like pharmaceuticals and lab use.

In an example, a high-quality plastic body may be combined with an inert glass-like inner surface to ensure chemical resistance. These are often used for sensitive medications requiring high stability and low interaction with the container. In another example, a multi-layer plastic container with protective barrier properties similar to glass may use advanced polymers with a glass-like barrier, thereby minimizing medication interactions, particularly in biologics and complex medications. In yet another example, polymer syringes and vials have properties similar to glass but are made from high-quality polymers that are highly resistant to medication interaction, thereby being suitable for pre-filled syringes, biologics, and sensitive medications.

The multi-layer plastic containers that are lined with protective materials to offer the inert properties of glass while using a plastic exterior are widely used in injectable medication delivery systems. Further, polymer syringes and vials with barrier properties to replicate the non-reactive qualities of glass are used in pre-filled syringes and other pharmaceutical applications. Such manufacturing processes focus on balancing the advantages of plastic (such as durability and weight) with the inert, non-reactive nature of glass to create safe storage options for sensitive substances.

Certain medications require specific storage conditions to maintain their efficacy. For example, some medications, such as insulin or blood products, require refrigeration, while others, like certain contrast agents, may need heating. The storage module 104 may include mechanisms for the refrigeration and/or heating of medications in a compartmentalized manner, ensuring that the medication fluids remain in optimal condition until the medication fluid is ready for use.

The system 102 may be preloaded with standard information about common medications) or customizable, allowing healthcare providers to tailor the information based on specific institutional needs, treatment protocols, or patient populations. The storage module 104 includes information on a wide range of IV medications, including name and type of medication, concentration and formulation, indications and contraindications, dosage guidelines, including patient-specific variables like age, weight, and medical condition, potential interactions and incompatibilities with other medications, warnings for controlled substances to ensure compliance with legal and safety requirements. The storage module 104 is integrated with automated verification systems to reduce errors in medication administration. For example, before a medication is administered, the system 102 may automatically check the patient's identity, medication type, and dosage against the prescription order. This feature is designed to prevent errors like incorrect dosing, administering the wrong medication, or giving medication to the wrong patient. Such systems are commonly used in hospitals with smart IV pumps, which pull data from the medication library to confirm that the dosage adheres to the hospital's guidelines.

The system 102 may include a digital repository containing detailed information about each medication stored in the physical library. It serves as the intelligence behind the physical system, ensuring that medications are administered safely and according to prescribed protocols. The storage module 104 is critical for automating processes, improving accuracy, and preventing human errors.

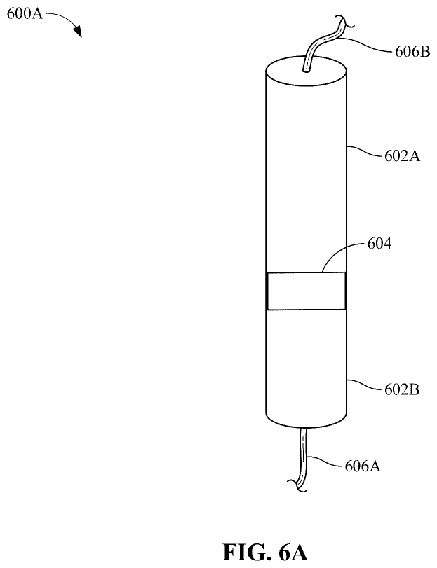

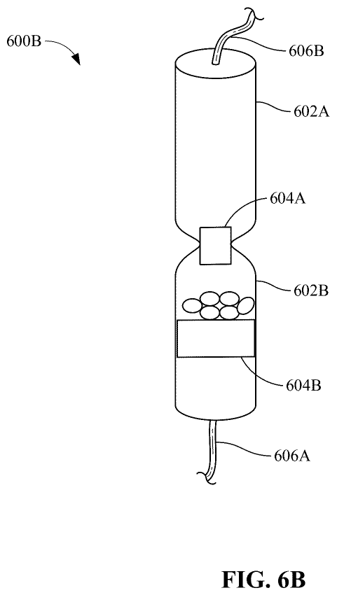

In an embodiment, the plurality of medication containers 104A may be designed as the multi-chamber container for mixing purposes. The multi-chamber container allows the separation of the medications and the diluents until required for use as discussed, for example, in FIG. 6A and FIG. 6B. The first chamber of the multi-chamber container may hold the medication in powdered form while the second chamber of a multi-chamber cartridge holds a liquid diluent. In another embodiment, a built-in mechanism applies pressure to move the separating stopper into the second chamber so that the powder and the liquid diluent may be mixed through operations like shaking, inversion, agitation, mechanical aspiration-dispense blending, vibratory mixing, vortex mixing, or sonication.

In an embodiment, the storage module 104A may also include a mixing chamber (not shown in FIG. 2). The mixing chamber is where the liquids and powders meet. The system 102 includes a mixing chamber that includes a motor-driven paddle, magnetic stirrer, or vortex mixer that blends the powders and liquids until homogeneous. Further, the system 102 includes a touch screen control panel that facilitates users to select which ingredients to mix, the ratios, and the mixing time. Further, the sensors may measure flow rates and quantities, ensuring accuracy for each component.

In an embodiment, the healthcare worker selects a desired mix of a treatment fluid from the touch screen (e.g., “Mix Liquid A with Powder B and Powder A”). In another example, a desired mix of the treatment fluid is determined based on the prescription data of the patient or the dosage data associated with the patient. The system 102 activates pumps that expel a precise amount of liquid from Tank A and direct it into the mixing chamber. Simultaneously, dispensers for Powder A and Powder B release specified quantities into the mixing chamber using the one or more carrier conduits 108. Once all ingredients are in the mixing chamber, the powder A and powder B are mixed using a mixing mechanism (such as a motor-driven paddle, magnetic stirrer, or vortex mixer). The system 102 ensures a thorough mixing by varying speed and mixing directions, ensuring no clumps of powder remain. The final mixture is dispensed through a nozzle or valve into a container using the patient conduit 110 for use.

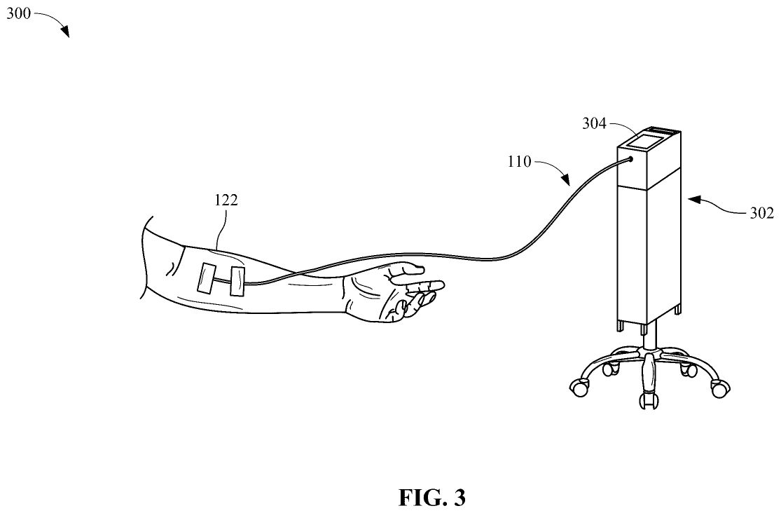

FIG. 3 illustrates a schematic diagram 300 of the system 102, in accordance with an embodiment of the disclosure. FIG. 3 is explained in conjunction with elements from FIG. 1A, FIG. 1B, and FIG. 2. The diagram 300 includes a cabinet 302 which encloses the elements of the system 102. The system 102 includes the storage module 104, the plurality of pressurizing pumps 106, the one or more carrier conduits 108, and the plurality of pressure valves 112. The storage module 104 includes the plurality of medication containers 104A. The system 102 may further include the plurality of filter modules 114, the sterilization module 116, and the dispensing pump 120. The system 102 includes a user-interface 304, and the patient conduit 110 exiting from the cabinet 302.

The system 102 may include the user-interface 304 for providing access to various features and data of the system 102. For example, the user-interface 304 may be an input/output interface in the form of a touch interface, a voice-enabled interface, a keypad, or a combination thereof. In an embodiment, the system 102 may leverage the use of the graphical user-interface 304 to verify biometric data associated with the patient 122 and/or a healthcare professional using the system 102. Further, the system 102 may employ the user-interface 304 to receive input from the healthcare professional (such as a nurse or doctor). The input may include, for example, patient-specific values that regulate the operation flow of the treatment fluid, prescription data, other patient data, and so forth. For example, the healthcare professional may interact with the system 102 using the user-interface 304. Further, the healthcare professional may input commands, monitor operations, and adjust settings through the user-interface 304. For instance, the healthcare professional may modify infusion rates or pause operations based on patient responses.

In an embodiment, the user-interface 304 provides healthcare workers with real-time data on stored medications, completed or pending treatments the patient based on prescriptions data, and other operational reports, such as an indication of blockages in conduits, replacement of filters, or any other abnormality in operation of any components of the system 102. Reports generated through this interface include medication preparation history, dosage administration records, and inventory updates. For example, the system 102 may analyze the detailed report to ensure the correct dosage and the preparation process for the medication delivery to the patient 122.

In an embodiment, each of the plurality of medication containers 104A may be labeled. Such labeling and marking of the medication containers facilitate accurate administration of the medication. In an embodiment, a first label is positioned on each of the plurality of medication containers 104A. Further, the system 102 may include a scanner or a sensor configured to read the first label of each of the plurality of medication containers 104A. Thereafter, the system 102 may retrieve the medication data associated with each of the plurality of medication containers based on the reading. Such scanning facilitates efficient tracking of the medication container 104A. Further, the labeling and marking of the medication containers provide patient safety by minimizing errors in medication delivery. For example, the system 102 may verify the biometric data associated with the patient or the healthcare professional and analyze the mediation data. This may facilitate to determine the dosage data and provide detailed information about the medications, such as their properties, dosing requirements, and safety protocols. Together, these components work synergistically to enhance the safety, efficiency, and accuracy of IV medication administration.

In an embodiment, the system 102 may be configured to identify the one or more medication containers 206 from the plurality of medication containers 104A based on the dosage data. The medication fluid from each of the one or more medication containers 206 from the plurality of medication containers 104A is combined to form the treatment fluid. Thereafter, the one or more carrier conduits 108 connects the one or more medication containers from the plurality of medication containers 104A to the patient conduit 110, which serves as the final delivery pathway to the patient. The carrier conduits 108 and the patient conduit 110 are equipped with pressure sensors, the plurality of pressurizing pumps 106 and the plurality of pressure valves 112. Further, the system 102 is configured to identify the one or more pressure valves from the plurality of pressure valves. The one or more pressure valves are associated with a connection between the identified one or more medication containers and at least one carrier conduit of the one or more carrier conduits 108, the at least one carrier conduit, or the patient conduit 110. Thereafter, the system 102 is configured to generate the set of control parameters for controlling each of the one or more pressurizing pumps 106 and the one or more pressure valves 112. The one or more pressurizing pumps 106 are associated with the identified one or more medication containers 206 from the plurality of medication containers 104A, and the set of control parameters indicates a pressure value and a flow rate.

In an embodiment, the system 102 may determine the pressure data based on the dosage data. The pressure data includes information associated with pressure gradient to regulate the fluid flow rate corresponding to the dosage and maintain a sterile environment. The pressure data includes a first pressure value associated with the one or more pressurizing pumps, a second pressure value associated with the connection between each of the one or more medication containers and the at least one carrier conduit, a third pressure value associated with the at least one carrier conduit, and a fourth pressure value associated with the patient conduit. The system 102 identifies the one or more medication containers from the plurality of medication containers 104A based on the dosage data, then generates the set of control parameters for the plurality of pressurizing pumps 106 and the plurality of pressure valves 112 to achieve precise delivery. The pressure gradient is maintained by ensuring that the first pressure value associated with the one or more pressurizing pumps is higher than the second pressure value associated with a pressurizing pump arranged between the medication container and the carrier conduit 108, which is, in turn, higher than the third pressure value in the carrier conduit and the fourth pressure value in the patient conduit 110. In an embodiment, the system 102 may provide accuracy and safety by identifying the correct medication containers and generating control parameters for the pressurizing pumps and pressure valves.

The system 102 adjusts pressure values at different points to prevent errors and maintain smooth medication fluid delivery to the patient 122. For example, the pressure in one of the plurality of medication containers 104A is kept higher than the pressure in the associated one or more carrier conduits 108, and the pressure in one or more carrier conduits 108 is kept higher than the pressure in the associated patient conduit 110. The step-by-step reduction in pressure prevents any backflow of medication in the medication delivery process.

In an embodiment, the system 102 may generate reports on various operations, such as medication preparation, dosage administration, and inventory updates, and also perform medication verification, patient verification, prescription verification, and/or provider verification. The system 102 may have a calculator, software system, or Artificial Intelligence (AI) module 118 that determines how much of each medication and fluid it contains and how much of each medication and fluid has been given when it was given, to whom it was given and the provider giving the medication. For example, the healthcare professional may use the system to check a report confirming that the correct dose was prepared and delivered to the patient. Such an inventory control and monitoring system may have safety systems for medication verification, patient verification, prescription verification and/or provider verification. For example, the system 102 may leverage the use of the graphical user-interface 304 to verify biometric data associated with the healthcare professionals (such as a nurse or doctor, thereby verifying the provider. The user-interface 304 may be password and/or biometric sensory protected. The user-interface 304 may allow the provider to enter the dosages and/or infusion rates for the medications and the fluids. The user interface 304 may allow visualization of the medication information discussed above. It may also provide visual and auditory information concerning medication delivery, fluid delivery, system states, safety information, alarm statuses, and/or any other information on the state of the system and/or its components.

In an embodiment, the system 102 detects a quantity of medication present in the medication container. For example, the system 102 may determine how full a medication container is, ensuring that healthcare providers are aware of the available medication volume. In an example, the system 102 may determine expiration data associated with the medication fluid or the treatment fluid, such that if the medication container A includes a medication fluid X set to be expired in 6 hrs. and another multi-chamber container B includes a treatment fluid that is a combination of medication fluid Y and Z set to expire in 2 hrs. the system 102 may determine the medication fluid to be expired sooner and generate the notification data associated therewith.

Further, the pressure sensors may be incorporated into the medication containers 104A to detect a presence of bubbles of air or gases, a critical safety feature as air embolism is a serious risk in IV administration. The medication container 104A is one state of container that is “full” with both a liquid and a gas region filled to a pressure of P_{full}. The medication container 104A may include a bladder or septa and a filling portal such as a rubber stopper that allows for separation between the air or gases and the medication and/or fluids. The medication container 104A may have a mechanism to reduce coring. For example, a value, luer lock, filter, or other device or combination of devices to allow connection of the container to a carrier conduit 108. The medication container 104A may include an isolated, separate air or gas-filled region and the filling portal for the gas on the top of the container. For example, the medication container 104A is “empty” which is one state of the container where the pressure P_{empty} is now lower than P_{full}. The difference in P is P_{Δ}=P_{full}-P_{empty}. The amount of fluid released into the carrier conduit may be calculated from the volume of the two regions in the container and the P_{Δ}. For example, if P_{full}was 50 PSI and P_{mpty}was 25 PSI then P_{Δ}would be 25 PSI and approximately half of the container's total volume has been unloaded. In another embodiment of the internal flow of fluids and medications, the flows could be controlled by pumps, valves, and filters that do not require pressure differences between components, other than a driving pressure and valves to keep the fluids moving in the proper direction. In an embodiment, when the medication fluid or the treatment fluid from the medication containers are not completely administered to the patient 122, the remaining medication fluid or the treatment fluid may not be discarded. However, the system 102 may control the storage module 104 to maintain the sterility of the remaining medication fluid or the treatment fluid for administering it to the next patient. In an embodiment, the patient conduit 110 is configurable to connect to a subsequent patient after the delivery of the treatment fluid to the patient. The system 102 is configured to deliver the treatment fluid or new treatment fluid to the patient 122.

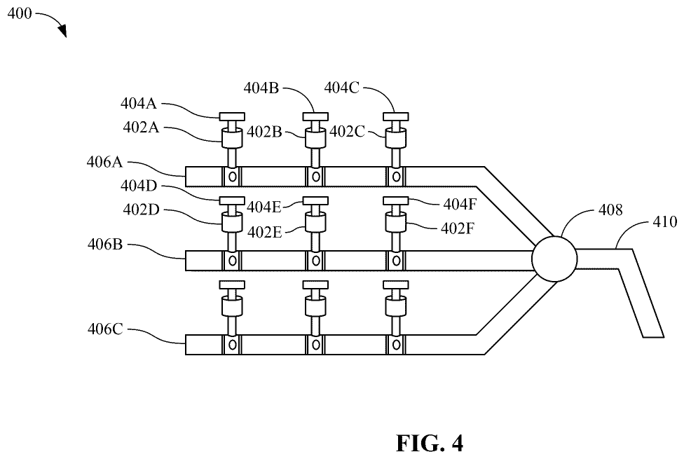

FIG. 4 illustrates a schematic diagram of conduits of the system 102, in accordance with an embodiment of the disclosure. FIG. 4 is explained in conjunction with elements from FIG. 1A-1B, FIG. 2 and FIG. 3. FIG. 4 includes a plurality of medication containers such as a first medication container 402A, a second medication container 402B, a third medication container 402C, a fourth medication container 402D, and a fifth medication container 402E. Each of the plurality of medication containers stores a distinct medication fluid. The plurality of medication containers is operatively associated with corresponding pressurizing pumps a first pressurizing pumps 404A, a second pressurizing pumps 404B, a third pressurizing pumps 404C, a fourth pressurizing pumps 404D, and a fifth pressurizing pumps 404E. Each of the plurality of pressurizing pumps is configured to apply controlled pressure to its corresponding medication container. Such a configuration between pressurizing pumps and medication containers facilitates precise extraction of the medication fluid from the corresponding medication container.

Further, each of the plurality of medication containers (402A, 402B, 402C, 402D, 402E, 402F) may be connected to one or more carrier conduits (such as but not limited to a first carrier conduit 406A, a second carrier conduit 406B, and a third carrier conduit 406C). Such an arrangement may form channels for transporting medication fluids to a mixing chamber 408. The mixing chamber 408 is configured to combine one or more medication fluids from at least one of the medication containers based on the dosage data. The mixed the medication fluid is directed through a patient conduit 410 which is connected to the mixing chamber 408 and is configured to deliver the treatment fluid to the patient 122. It is to be noted that the system 102 includes the medication container(402A, 402B, 402C, 402D, 402E, 402F), the pressurizing pumps (404A, 404B, 404C, 404D, 404E, 404F), the one or more carrier conduits (406A, 406B, 406C), the mixing chamber 408, the patient conduit 110 however, the disclosure may not be so limiting, and the system 102 may include fewer or more components to perform the same or other functions associated therewith.

Some medications, particularly those with high viscosity or specific delivery requirements, may require pressurization for proper administration. The medication containers in the storage module 104 may be pressurized by gas, hydraulic, or mechanical means to ensure smooth and controlled flow into the patient's circulatory system, specifically applicable in applications like blood transfusions or when using high-flow infusion systems. For example, lyophilized medications (freeze-dried) may be separated from a saline or sterile water solution until reconstitution is needed. Pressurizing pumps, such as gases or hydraulic fluids, may also be stored in separate compartments to maintain the integrity of the medication. The storage module 104 facilitates the movement and selection of individual containers or groups of containers, allowing the system 102 easy access to medications. Further, the system 102 may facilitate the movement of the containers to the IV pump, ensuring a seamless workflow and enabling fast response times in high-pressure environments such as operating rooms and ICU's. The system 102 includes mechanisms for measuring and maintaining pressure in the containers, and it may also handle depressurization as needed, thereby ensuring that medications are delivered at the correct pressure, preventing over-infusion or under-infusion. For example, the pressurizing pumps may include a peristaltic (including hose, tube, or other types), diaphragm, centrifugal, impeller, rotary lobe, screw, or other types of pumps.

In an embodiment, the system utilizes different types of pressurizing pumps to ensure efficient and precise fluid handling. The plurality of pressurizing pumps may include one or more peristaltic pumps configured to apply pressure to their corresponding medication container using compressible flexible tubing. For example, the medication is stored in the medication containers (402A, 402B, 402C, 402D, 402E, and 402F), and the peristaltic pump gently compresses the tubing to create a controlled flow of the medication. The peristaltic pump mechanism may provide accurate medication dosage delivery without direct contact between the pressurizing pump and the medication fluid and minimizing the risk of contamination of the medication.

In another embodiment, each pressurizing pump may correspond to hydraulic pumps, designed to apply pressure to the corresponding medication containers (402A, 402B, 402C, 402D, 402E, and 402F) using a liquid medium. For example, if the medication containers (402A, 402B, 402C, 402D, 402E, and 402F) contain a lyophilized medication and a liquid diluent, the hydraulic pump injects a precise volume of liquid into the medication containers (402A, 402B, 402C, 402D, 402E, and 402F) to raise the pressure inside the medication container (402A, 402B, 402C, 402D, 402E, and 402F). The increment of pressure inside the medication containers (402A, 402B, 402C, 402D, 402E, and 402F) triggers the release of the separation barrier, see FIG. 6B, allowing the medication and diluent to mix thoroughly before delivering to the patient 122.

In an embodiment, each pressurizing pump includes an alternative type of pump, selected from a diaphragm pump, piston pump, or rotary vane pump, configured to pressurize the medication container or multi-chamber container for delivering the medication fluid or solubilized medication.

In an embodiment, each of the plurality of pressurizing pumps 106 corresponds to compressed gas pumps to generate the pressure necessary for delivering the medication fluid or solubilized medication. In an embodiment, each of the plurality of pressurizing pumps 106 is configured to apply the pressure for the corresponding medication container of the plurality of medication containers using a compressed gas.

In another embodiment, each of the plurality of pressurizing pumps 106 corresponds to peristaltic pumps configured to create the pressure necessary for delivering the medication fluid or solubilized medication by compressing flexible tubing. For example, each of the plurality of pressurizing pumps 106 is configured to apply the pressure for the corresponding medication container of the plurality of medication containers using a compressible flexible tubing.

In an embodiment, each of the plurality of pressurizing pumps 106 corresponds to hydraulic pumps configured to pressurize the medication container or multi-chamber container using a liquid medium to generate the required pressure. For example, each of the plurality of pressurizing pumps 106 is configured to apply the pressure for the corresponding medication container of the plurality of medication containers using a liquid medium.

The system 102 may further include a plurality of pressure valves 112 to regulate the flow of fluids within the one or more conduits. The system 102 may dynamically control the plurality of pressure valves 112 to control a flow of the medication fluid from the medication container (402A, 402B, 402C, 402D, 402E, or 402F) towards the patient conduit 410. In an embodiment, the system 102 may identify the one or more medication containers (402A, 402B, 402C, 402D, 402E, and/or 402F) from the plurality of medication containers 104A based on the dosage data. The medication fluid from each of the one or more medication containers is combined to form the treatment fluid. For example, the system 102 may receive and analyze the medication data (e.g., fluid type, properties, and storage conditions) and the patient data (e.g., medical prescriptions, body weight, and treatment requirements). Based on the analysis, the system 102 generates the dosage data, specifying the concentration and volume of medication fluids to be combined into the treatment fluid. For example, the plurality of pressure valves 112 may have piezo, piezoelectric, solenoid, hydraulic, pneumatic, and/or other types of valves.

The system 102 further generates a set of control parameters for operating the pressurizing pumps 106 and the pressure valves 112. The set of control parameters may provide precise control of the flow rates and mixing ratios required for the treatment fluid. The pressurizing pumps (404A, 404B, 404C, 404D, 404E, and/or 404F) may adapt to the unique properties of each medication fluid, ensuring consistent delivery despite variations in viscosity, density, or temperature sensitivity. The pressure valves 112 provide additional fine-tuning to maintain optimal flow conditions across the conduits.

The system 102 integrates seamlessly with real-time data inputs and operates dynamically to accommodate changing treatment requirements. For example, if patient data indicates a need for a change in medication dosage, the system 102 may dynamically adjust the set of control parameters, thereby modifying the pressure of the pressurizing pump 106 and the pressure valve 112 settings to control the flow of the treatment fluid that matches the new dosage requirements. For example, the healthcare worker may input the required dosage requirement, thereafter the system 102 may dynamically adjust the set of control parameters to control the flow of the medication fluid.

In an embodiment, when multiple medication fluids are required to be administered simultaneously or sequentially, the system 102 may control a flow of each of the medication fluids based on the dosage data. For example, where the patient 122 needs a combination therapy involving multiple medications, the system 102 combines fluids stored in first medication container 402A and the second medication container 402D within the mixing chamber 408 to deliver a customized infusion via the patient conduit 410. Such a modular design enables compatibility with a variety of medication types, including liquids, suspensions, or reconstituted powders, making the system 102 versatile for diverse therapeutic applications.

In an embodiment, the system 102 may leverage the use of AI model 118 to optimize dosage data calculations and generation of the set of control parameters. The AI model 118 may be trained on datasets that include historical patient data, medication properties, and clinical guidelines, potentially utilizing multiple hidden layers to capture complex interactions between input variables. The use of such computational models enhances the system's ability to deliver accurate and personalized treatments, thereby improving patient outcomes while maintaining high standards of safety and efficiency.

Referring to FIG. 4, there is a schematic diagram of the internal flow of medication fluids. For example, a pressure in the first medication container 402A is P3, and the pressure in the valves and filters that lead from the first medication container 402A to the intermediate carrier conduit 406A is P3a. Further, the pressure in the intermediate carrier lines 406B is P2 and the pressure in the secondary valves and filters is P2a. Similarly, the pressure in the patient conduit 110 is P1. In an example, the pressure in the patient conduit 110 may raise as high as 250 mmHg on average and up to 500+ mmHg during peaks and discusses the need for multiple pressure sensing valves to prevent back flow. Further, the average pressure is highest in P3 then P3a then P2 then P2a, and then P1.

In an embodiment, the plurality of medication containers 104A includes one or more sets of medication containers, such that one or more medication containers in a set of medication containers are connected in a series configuration with one of the one or more carrier conduits, and each set of the one or more sets of medication containers is connected in a parallel configuration with the one or more carrier conduits 108. The diagram shows that a plurality of medication containers may be arranged in series and/or in parallel. Medication fluids that are compatible may be arranged in series. Medication fluids that are incompatible may be arranged in parallel. The secondary valves and filters may combine compatible medications. The secondary valves and filters may keep incompatible medications separated. The patient line may combine compatible medications. The patient line may keep incompatible medications separated. The patient line may have more than one channel. The patient line may have a “STAT” low volume, high flow rate channel. In another embodiment of the internal flow of fluids and medications, the flows could be controlled by pumps, valves, and filters that do not require pressure differences between components, other than a driving pressure and valves to keep the fluids moving in the proper direction.

In an embodiment, the storage module 104 includes information on medication compatibility. If two incompatible medications are prescribed together, the system may flag this issue and alert the healthcare provider before the medications are mixed or delivered. This is crucial for preventing adverse medication reactions and IV-line blockages.

The system 102 collects real-time data during the administration process, including flow rates, pressure levels, and air bubble detection alerts. Such information may be recorded and used for compliance reporting, tracking outcomes, and identifying any deviations from the prescribed treatment plan. In systems used in high-risk areas like ICUs, this kind of data is vital for ensuring patient safety and providing clinicians with detailed insights into the medication administration process. The storage module 104 may be fully integrated with a hospital's electronic medical records (EMR) system, allowing seamless access to patient data, including real-time vital sign and other patient data, historical medication records, and ensuring continuity of care. Such integration supports automated checks, such as ensuring that the medication being administered matches the patient's prescription and reduces the likelihood of human errors. For example, in some hospitals, smart IV pumps equipped with both physical and data libraries are standard. The physical library stores medications in specialized containers, while the data library is continuously updated with the latest medication information. When the healthcare provider initiates an infusion, the data library verifies that the correct medication is selected, checks the dosage, and ensures that the flow rate is correct, all while tracking real-time data like pressure and air bubble detection. The system 102 is crucial in avoiding medication errors, especially in critical care settings.

While the specifications contain many specifics, these should not be construed as limitations on the scope of the claims or of what may be claimed, but rather as descriptions of features specific to these embodiments or methods. Certain features that are described in this specification in the context of separate embodiments or methods may also be implemented in combination in a single embodiment or method. Conversely, various features that are described in the context of a single embodiment or method may also be implemented in multiple embodiments or methods separately or in any suitable sub-combination. Moreover, although features may be described above as acting in certain combinations and even initially claimed as such, one or more features from a claimed combination may in some cases be excised from the combination, and the claimed combination may be directed to a sub-combination or a variation of a sub-combination. Similarly, while operations are depicted in the drawings in a particular order, this should not be understood as requiring that such operations be performed in the order shown or in sequential order, or that all illustrated operations be performed, to achieve desirable results. Although embodiments of various devices and methods are described herein in detail with reference to certain versions, it should be appreciated that other versions, embodiments, methods of use, and combinations thereof are also possible. Therefore, the spirit and scope of any claims should not be limited to the description of the embodiments or methods contained herein.

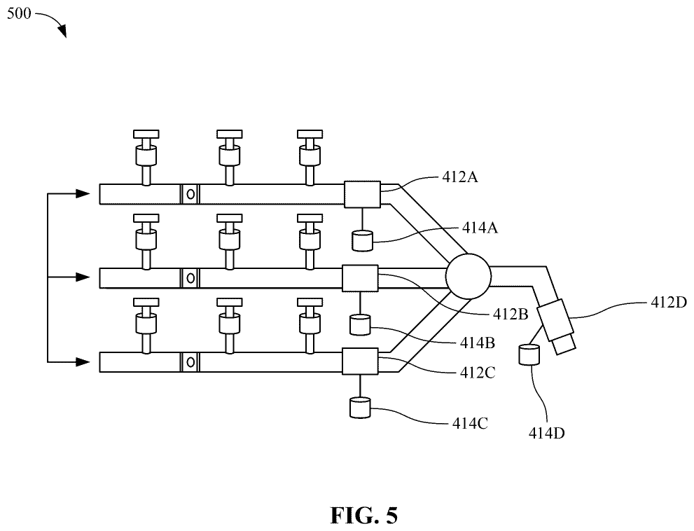

FIG. 5 illustrates another schematic diagram of the conduits of the system 102, in accordance with an embodiment of the disclosure. FIG. 5 is explained in conjunction with elements from FIG. 1A-1B, FIG. 2, FIG. 3, and FIG. 4. The diagram 500 depicts the system 102. FIG. 5 includes a plurality of medication containers such as the first medication container 402A, the second medication container 402B, the third medication container 402C, the fourth medication container 402D, and the fifth medication container 402E. Each of the plurality of medication containers stores a distinct medication fluid. The plurality of medication containers is operatively associated with corresponding pressurizing pumps the first pressurizing pumps 404A, the second pressurizing pumps 404B, the third pressurizing pumps 404C, the fourth pressurizing pumps 404D, and the fifth pressurizing pumps 404E. Each of the plurality of pressurizing pumps is configured to apply controlled pressure to its corresponding medication container. Such a configuration between pressurizing pumps and medication containers facilitates precise extraction of the medication fluid from the medication container. The system 102 further includes the one or more carrier conduits (406A, 406B, 406C), the mixing chamber 408, and the patient conduit 410, described for example, in FIG. 1.

The plurality of medication containers 402 may store different medication fluids or solutions required for patient treatment. Each medication container is connected to a corresponding pressurizing pump. For example, the first medication container 402A is connected to the corresponding first pressurizing pump 404A, the second medication container 402B is connected to the corresponding second pressurizing pump 404B, and so forth. The pressurizing pumps 404 applies controlled pressure to transfer the medication fluid in the corresponding medication container from the respective container into a designated carrier conduit. For example, the first pressurizing pump 404A applies pressure to control the transfer of medication fluid in the first medication container 402A to the carrier conduit 406A. In a similar manner, another medication fluid(s), say the medication fluid from the medication container(s) 406D may also be transferred to corresponding carrier conduit 406B based on a pressure from the fourth pressurizing pump 404D applied thereto. In such a case, the medication fluid, say the first fluid from the first medication container 402A, and the medication fluid, say second fluid from the fourth medication container 402D are mixed, for example, stirred or shaken for a predefined period of time and at a predefined intensity to prepare the treatment fluid. The concentration or quantity associated with the first fluid and the second fluid is determined from the dosage data. Accordingly, the first pressurizing pump 404A and the fourth pressurizing pump 404D along with other pressure valves, filters, and other components of the system 102 may be controlled for controlling the flow of the first fluid and the second fluid to a corresponding carrier conduit and the mixing chamber 408.

In an embodiment, the carrier conduits 406 merge into the mixing chamber 408. The mixing chamber 408 facilitates the delivery of the treatment fluid to the patient. Between medication fluids of different types, a separator solution may be introduced to serve as a buffer, preventing cross-contamination or unintended chemical interactions during the infusion process.

In an embodiment, the system 102 may further one or more filter modules (such as a first filter 412A, a second filter 412B, a third filter 412C, and a fourth filter 412D) and a sterilization module (such as a first sterilization module 414A, a second sterilization module 414B, a third sterilization module 414C, and a fourth sterilization module 414D), and a dispensing pump 120. In an embodiment, a filter and sterilization unit 414 is positioned downstream of the mixing chamber 408. The one or more filter modules 412 may perform functions such as removing particulates, contaminants, or microorganisms from the treatment fluid. Further, the sterilization module is arranged in connection with each of the plurality of filter modules 412, configured to periodically sterilize each of the plurality of filter modules. The combination of the one or more filter modules and the sterilization module may uphold sterility and fluid quality during delivery. Further, the one or more filter modules and the sterilization module enhance the safety and reliability of the system 102, especially for applications requiring sterile delivery. For example, the one or more filter modules 114 may have one-way valves, filters, microfilters, and/or other types of valves and/or filters.

For example, when the first fluid from the first medication container 402A and the medication fluid, say the second fluid from the fourth medication container 402D are mixed from 402A and 402D to form the treatment fluid. This treatment fluid will be temporarily stored in the mixing chamber 408 or the dispensing pump 120 before a movement of the treatment fluid from the mixing chamber 408 to the patient conduit 410, and subsequently to the patient 122. During the operation, the one or more filter modules 114 may perform filtering of the treatment fluid. Thereafter, the sterilization module is configured to sterilize each of the one or more filter modules using the UV light. In an example, if the treatment fluid is left in mixing chamber 408 then the system 102 may generate the set of control instruction to hold the sterilization, otherwise, it will get sterilized.

In an embodiment, the each of the plurality of filter modules is arranged in connection with the one or more carrier conduits 108, or the patient conduit 110. Further, the each of the filter modules includes but is not limited to a microbe filter or blood cell filter. In an embodiment, a pressure valve is associated with each of the filter modules to prevent cross-contamination, backflow and backdiffusion of one medication by another medication in the system during the operation of the filter module. The system 102 may include microbe filters, pressure valves, one-way pressure valves, drip chambers, and/or medication/molecular detection systems. The one-way pressure valves of the filter modules ensure that fluids only flow in one direction, preventing contamination, backflow, and backdiffusion in the filter modules which could introduce pathogens or incompatible medications into the patient's system.

The microbe filter may filter one or more microbe particles. For example, the microbe filter removes particulate matter or microorganisms from the medication before it enters the patient's bloodstream, enhancing safety and reducing the risk of infections or adverse reactions. In an embodiment, each filter module is positioned along, for example, but not limited to, the patient conduit 110, the one or more carrier conduits 108, or a connection between the medication container 104A and the one or more carrier conduits 108. Each filter module 412 includes a microbe filter (such as a bacterial and viral filter) configured to prevent bacterial and viral particles from contaminating the treatment fluid as well as medication fluids stored in the medication containers 402. Each filter module 412 further includes a blood cell filter configured to filter out blood cells or blood-particulate matter from the treatment fluid as well as medication fluids stored in the medication containers 402. The microbe filter is selected based on pore size specifications to capture particles smaller than 0.3 microns, and the blood cell filter is selected to filter particles larger than 6 microns. Further, the one or more filter modules 412 are replaceable and include labels or electronic identifiers for tracking filter usage, condition, and expiration dates. In an embodiment, a second label is positioned on each filter module 412 of the plurality of filter modules 412. Further, the scanner is configured to read the second label of each of the plurality of filter modules 412. Thereafter, the system 102 retrieves the filter data associated with each of the plurality of filter modules 412 based on the reading. System 102 may include disposable or non-disposable components (not shown for the sake of brevity). The disposable components (single use) could be used in high-risk areas such as those in direct contact with fluids, ensuring sterility and reducing cross-contamination between patients. The non-disposable components (re-usable) could be designed for long-term use with easy-to-clean and sterilizable materials.

Further, the pressure sensors could monitor the flow characteristics of the system 102 in real-time, detecting issues such as blockages, leaks, or abnormal pressure. This would provide healthcare providers with immediate feedback and prevent potential complications such as extravasation (fluid leaking into surrounding tissue) or dosage errors. Further, the sterilization module may include disposable and non-disposable elements including, but not limited to pressure valves, one-way flow valves, filters, microfilters, UV lights, and/or other sterilization systems.

Further, the sterilization module performs periodic sterilization of the one or more filter modules 412 without requiring manual intervention. For example, the sterilization system uses ultraviolet (UV) light or chemical agents to ensure continuous operational efficacy.

In an embodiment, the system 102 is configured to monitor filter integrity by detecting flow anomalies or pressure changes across each of the one or more filter modules 412. Thereafter, upon detecting reduced filter efficacy, the system 102 is configured to generate notification data for filter replacement or maintenance. For example, the treatment fluid may be passed through one or more filter modules 412. The filter modules 412 prevent contamination by removing bacterial and viral particles by filtering out blood cells and blood-borne particulates. The system 102 may be configured to monitor the filter performance (performance data) during fluid infusion based on flow rate and pressure data and generate alerts (notification data) if contamination is detected or filter replacement is required.

Thereafter, the dispensing pump 120 is used to regulate the flow rate of the treatment fluid delivered through the patient conduit 410. The dispensing pump 120 may provide precise control over the infusion, thereby ensuring accurate adherence to the treatment protocol. The system 102 may leverage the use of the AI model 118 to enhance automation and dynamic control.

The AI module 118 interacts with the system 102 and processes input data such as medication data, patient-specific information, including real-time patient vitals data, and operational parameters from the pressurizing pumps, pressure valves, and conduits. The AI module 118 may analyze the input data to generate optimized control parameters, such as adjusting pressures of pressurizing pumps 106, and pressure valves 112 for controlling flow rates for delivering the treatment fluid to the patient in accordance with prescribed requirements. Furthermore, the AI module 118 may support learning models trained on historical patient data or clinical protocols, enabling predictive adjustments and fine-tuned control over the infusion process.

In an embodiment, the system 102 may include a pump safety monitoring system including a motor control system, an air-in-line system, a flow-stop detection system, a pressure sensing system, a mechanism position sensor (for example, whether doors are closed, disposable elements are installed correctly) and/or a total volume/total dose infused monitor. In an example, when the system 102 corresponds to a hemodialysis machine, the treatment fluid may include blood that flows through multiple filters and one-way valves to prevent contamination and ensure a safe return to the patient 122. In another example, when the system 102 corresponds to a modern insulin pump, the system 102 may use flow sensors and alarms to ensure that correct dosage is delivered consistently and accurately to the patient 122 over time. These types of technologies and principles are directly applicable to improving IV medication delivery systems. By adopting these advanced safety mechanisms and building on the success of existing IV fluid delivery pumps, and pressurized anesthetic gas and vapor delivery systems, the proposed IV medication system 102 significantly reduces errors, improves patient outcomes, and makes the process of IV medication administration safer and more efficient.

FIG. 6A illustrates a schematic diagram of a multi-chamber container 600A storing medication fluid, in accordance with an embodiment of the disclosure. FIG. 6A is explained in conjunction with elements from FIG. 1A-1B, FIG. 2, FIG. 3, FIG. 4, and FIG. 5. The diagram illustrates a multi-chamber container 600A. The multi-chamber container 600A may include two chambers. For instance, one chamber may hold a prepared treatment fluid while the other chamber is being prepared or filled, ensuring continuous delivery without interruptions. The multi nature refers to having powdered, lyophilized, evaporated, agglomerated, granulated, in one chamber and a diluent in another, separated for example by a rubber stopper. When the pressure pushes the stopper into the powder medication chamber and allows the diluent to flow in.

In an embodiment, the multi-chamber container 600A is incorporated to enable operational flexibility that allows simultaneous preparation of different treatment fluids or alternation between fluids during a prolonged infusion process. The multi-chamber container 600A incorporates two separate chambers, labeled as a first chamber 602A and a second chamber 602B configured to store different entities independently. A plunger 604 is positioned within the container to regulate the flow of the medication out of chamber 602A and to keep the pressurized contents in chamber 602B from entering chamber 602A. The first chamber 602A serves as a storage area for a first entity such as a medication or an additive, but not limited to, a chemotherapy medication, a blood thinner, or another pharmaceutical compound. The second chamber 602B, on the other hand, is where the pressurized fluid, such as, but not limited to, a gas, a liquid, a Newtonian fluid, a non-Newtonian fluid is stored to increase the pressure in the multi-chamber 600A system.

In one embodiment, the second chamber 602B containing the pressurized fluid, such as, but not limited to, a gas, a liquid, a Newtonian fluid, or a non-Newtonian fluid may be connected to a pressure line-in 606A (from the pressurizing pump). When the pressure inside the second chamber 602B increases, the plunger 604 (for example, a stopper) moves away from the pressure towards the first chamber 602A, thereby increasing a pressure inside the first chamber 602A containing the medication fluid. This may result in flowing of the medication fluid through a carrier conduit 606B.

FIG. 6B illustrates a schematic diagram of a multi-chamber container 600B storing medication fluid, in accordance with an embodiment of the disclosure. FIG. 6B is explained in conjunction with elements from FIG. 1A-1B, FIG. 2, FIG. 3, FIG. 4, FIG. 5, and FIG. 6A. The diagram illustrates a multi-chamber container 600B. As shown, the multi-chamber container 600B may include the first chamber 602A, the second chamber 602B, and the mixing mechanism (including an arrangement of a first plunger 604A, a second plunger 604B). In an example, the mixing mechanism may be arranged within the multi-chamber container 6008B. In another example, the mixing mechanism may be present external to the multi-chamber container 600B. In such a scenario, the mixing mechanism may include the application of ultrasound, shaking, inverting, agitating etc.

Pursuant to the present example, a design of the multi-chamber container 600B may include a narrowing of the chamber 602B in which the first plunger 604A is arranged or positioned. The first plunger 604A acts as a dividing stopper between the first chamber 602A and the second chamber 602B. Further, the second plunger 604B is arranged within the second chamber 602B narrowing. In operation, an upper portion of the second chamber 602B may contain a solid medication, and the first chamber 602A may contain a diluent. The second chamber 602B may be connected to a pressure line-in 606A (from the pressurizing pump). When a pressure inside the second chamber 602B increases, the second plunger 604B (for example, a stopper) moves towards the first plunger 604A, thereby making it move away from the pressure towards the first chamber 602A. As a result, the solid medication from the second chamber 602B moves towards the first chamber 602A and the plunger 604A becomes dislodged and falls into chamber 602A. This may result in mixing of the solid medication into the diluent, thereby forming the treatment fluid. Further, a pressure inside the first chamber 602A, now containing the treatment fluid, may ensure the flow of the treatment fluid through a carrier conduit 606B towards the patient.

The multi-chamber 600B is particularly beneficial in scenarios requiring complex treatment protocols, such as but not limited to, antibiotics, and/or chemotherapeutics, where lyophilized medications must be delivered accurately and in a timely manner. By combining mechanical functionality with advanced control mechanisms, the multi-chamber container 600B provides flexibility and safety in medication delivery.

In an embodiment, the multi-chamber container 600B allows for the pre-mixing of the first entity of the first medication chamber 602A and the second entity of the second medication chamber 602B under controlled conditions. The plunger 604 is actuated to combine the fluids in precise proportions inside the container, creating a ready-to-use formulation of the treatment fluid. This is useful in emergencies where time-sensitive treatment is required. For example, the system 102 generates control parameters for preparing a pre-mixed solution, i.e., the treatment fluid, of saline and blood thinner in less than 30 seconds, ready for immediate infusion. The system 102 further controls the flow of the treatment fluid that complies with prescribed concentrations and dosages.

The multi-chamber container 600A and the multi-chamber container 600B create a seamless and automated approach to IV medication administration. Together, these components significantly reduce errors, improve patient safety, and enhance overall efficiency in medical treatment settings. The system 102 may deliver single doses, multiple doses, continuous infusions, and/or discontinuous infusions. The system 102 may ensure medication and fluid compatibility. The system 102 may have more than one channel for fluid and/or medication delivery. Further, the system 102 may have a dose rate calculator to generate the dosage data. For example, the system 102 may have a dilution calculator that determines how to deliver discontinuous infusions of multiple medications, even for medications that would be otherwise incompatible if they were delivered simultaneously. The system 102 may contain disposable and/or non-disposable elements. The system 102 may have a small volume channel that would allow the rapid (i.e. “STAT”) delivery of medications to the patient.

A multi-chamber container 600B may include a first chamber that stores a powdered medication, a second chamber that stores a liquid medication or solvent, and a mixing mechanism within the cartridge. The system 102 may combine the powder from the first chamber and the liquid from the second chamber to form a treatment fluid, activate pressurization of the combined treatment fluid to match the operational requirements of the system, control parameters generated by the processor to initiate and regulate the mixing process and adjust pressure levels post-mixing to ensure seamless integration with the overall flow of treatment fluid to the patient. For example, a powder is stored in a first chamber and a liquid is stored in a second chamber of a multi-chamber container. The system 102 may initiate the mixing of the powder and liquid upon receiving dosage data and regulating the pressure within the mixed cartridge to match predefined system pressure levels for delivery.

In the pharmaceutical industry, the system 102 may be configured to control the mixing mechanism in order to mix active pharmaceutical ingredients, which might be in powdered form, with various liquid solvents. The system 102 uses precise dosing systems for both powders and liquids, with highly controlled environments to ensure accuracy and cleanliness. In an example, to effectively mix lyophilized medication powders with diluent, particle degradation needs to be minimized, and medication efficacy needs to be maintained. In an example, the mixing mechanism is implemented using a vortex mixer that may help with even mixing of the medication and the compatible diluent. In an example, the compatibility verification with the medication guidelines is necessary, as some powders may be damaged by aggressive mixing. For example, the system 102 may include a robotic arm to mix active pharmaceutical ingredients. Such as sensitive compounds, the system 102 may control the robotic arm to perform gentle swirling of the compound without vigorous shaking to dissolve the powder. Then, tilt the vial at an angle and gently rotate it to allow the diluent to wash over the powder, encouraging it to dissolve without vigorous shaking. Further, inject the diluent slowly and at an angle to reduce the formation of bubbles and help disperse the powder more evenly throughout the solution. After reconstitution, rolling the vial helps to mix without causing foaming or degradation, especially useful for protein-based medications that are prone to denaturation. If clumps form, a sterile needle is used to carefully break them up. Some medications reconstitute better at specific temperatures. The system 102 is configured to check the medical data indicating mixing instructions for bringing the diluent to room temperature before reconstitution to aid dissolution for certain powders.

Each medication may have unique requirements, so consulting the manufacturer's instructions for the specific reconstitution method is critical for maintaining medication efficacy and safety. Further, ultrasound (or sonication) may be used to mix lyophilized powdered medications with a diluent. Ultrasound waves may create rapid vibrations and cavitation (formation and collapse of microbubbles), which improve the mixing process by breaking up particles and enhancing solvent penetration. This is particularly useful when dealing with delicate or slow-dissolving powders, as ultrasound may speed up dissolution without excessive agitation. In a medical setting, though, the use of ultrasound is often limited due to concerns about potential heat generation or unintended chemical alterations. So, while it is feasible, it's crucial to ensure that the specific medication being reconstituted may safely undergo ultrasonic treatment.

In an embodiment, a solid dispersion or film casting is used to enhance the solubility of medication fluid by producing them in thin films or small, rapidly dissolving disks. Solid dispersion or film casting involves dispersing the medication entity in a soluble matrix (like a polymer) and then casting the medication into thin medication films or pressing the medication into small medication disks. When exposed to a solvent (like water), these medication films or medication disks dissolve quickly into a solvent, releasing the powder medication entity with an increased surface area, which facilitates faster and more complete dissolution of medication. For example, the techniques include flash drying which dries the solution containing the medication entity into a thin film, forming easily dissolvable material. Freeze casting is specifically for producing porous structures that may be compressed into films or disks for rapid dissolution. Such approaches are especially useful in pharmaceuticals, where rapid and enhanced solubility of active ingredients is desired for quick absorption in the body.

Although the present disclosure describes the medication containers 104A to have a multi-chamber or a multi-chamber, this should not be construed as a limitation. In other embodiments, some or all of the medication containers 104A may have single chamber, four chambers, or more. For example, in case of three chambers, a first chamber may store powdered medication, a second chamber may store a solvent, and a third chamber is applied with pressure for enabling mixing of the entities in the first chamber and the second chamber.

In addition to these techniques, granulation and agglomeration are commonly employed to improve the physical properties of the dry medication. Granulation, which may be conducted as either wet or dry granulation, is the process of combining fine the medication powders into larger, cohesive particles often with a binder. Wet granulation involves the use of a liquid binder to form a paste of medication powder, which is subsequently dried to create granules. Dry granulation, in contrast, compresses the medication powder directly into larger particles without the use of liquid, making it a preferred choice for medications sensitive to moisture. Agglomeration, a similar but distinct process, involves encouraging medication powders to form stable, chunk-like clusters through the controlled addition of moisture or the application of mechanical force. Agglomeration enhances the flow properties and stability of the final medication product while maintaining medication dissolution characteristics. Each method is chosen based on the stability, sensitivity, and desired physical properties of the medication, as well as the scale and cost-effectiveness needed for production.

Each technique for the mixing mechanism is selected based on the physical and chemical stability of the medication, its sensitivity to processing conditions, the desired characteristics of the final medication product, and the scalability and cost-effectiveness required for the production of medication. These techniques are crucial for ensuring the safe, efficient, and effective delivery of medications to patients, as they address critical factors such as handling, stability, and compatibility with the intended delivery systems.

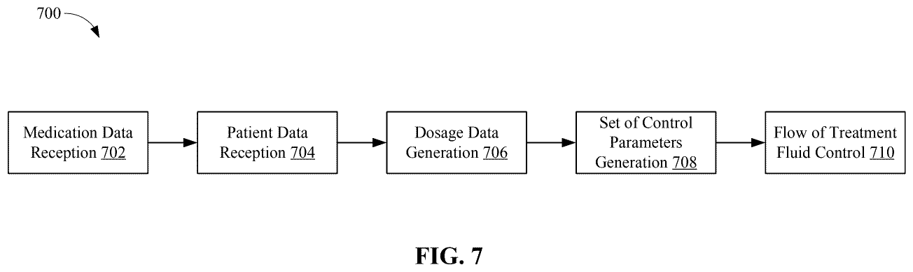

FIG. 7 illustrates a block diagram of exemplary operations for controlling medical fluid infusion for patients, in accordance with an embodiment of the disclosure. FIG. 7 is explained in conjunction with elements from FIG. 1A, FIG. 1B, FIG. 2, FIG. 3, FIG. 4, FIG. 5, and FIG. 6A-6B. With reference to FIG. 7 there is shown a flowchart 700. The operations of the exemplary method may be executed by any computing system, for example, by the system 102 of FIG. 1. The operations of the flowchart 700 may start at 702.

At 702, a medication data reception operation is performed. In an embodiment, the system 102 is configured to receive medication data associated with the medication fluid stored in each of the plurality of medication containers 104A. The medication data may be stored in a database or a storage unit associated with the system 102. The system 102 may be configured to receive the medication data from the storage unit. The medication data for a particular medication fluid in a medication container may include details such as the type of the particular medication fluid, the concentration of the particular medication fluid, and the volume of a particular medication fluid, which is stored in multiple medication containers within the system. For example, the system retrieves information that container A holds saline solution, container B holds an antibiotic at a concentration of 500 mg/mL, and container C holds a pain relief medication at 250 mg/mL. The system receives medication data, which includes information such as but not limited to, expiry date, handling instructions, recommended dosage, manufacturing details, and batch details associated with the medication fluid. For example, the system detects that a saline having a concentration of 0.9% is stored in the first medication container 402A, a blood thinner having a concentration of 2 mg/mL is stored in the second medication container 402B, and a chemotherapy medication having a concentration of 2 mg/mL) is stored in the third medication container 402C. Similarly, medication data includes information associated with the medication fluid such as saline (0.9%) in the fourth medication container 402D, a pain-relief medication (10 mg/mL) in container 402E, and an antibiotic (20 mg/mL) in the fifth container 402F.

At 704, a patient data reception operation is performed. In an embodiment, the system 102 is configured to receive patient data associated with the patient 122. The patient data includes prescription data and real time patient vitals data. The patient data may include, but is not limited to, personal information, test results, insurance information, medical condition, treatment history, patient's medical history, health parameters, vital signs, and the prescription data that specify the required current treatment. For example, the patient data indicates that the individual weighs 70 kg, has a renal condition, and requires an antibiotic dosage of 15 mg/kg along with a pain relief infusion at a rate of 5 mL/hour. The prescription data includes treatment instructions, such as but not limited to, dosage requirements, delivery rates, and duration. In an example, the prescription for the patient 122 specifies the need for a blood thinner at 2 mg/hour, a chemotherapy medication at 1 mg/hour, and saline as a carrier fluid at 10 mL/hour. In another example, the prescription data indicates a pain-relief medication at 5 mg/hour, an antibiotic at 10 mg/hour, and saline as a carrier fluid at 15 mL/hour.

In an example, the prescription data is obtained in a secure manner, such as while following rules that ensure that healthcare providers that provide the prescription data are licensed to do so and that the healthcare providers that deliver the mediations (if not the same as the prescriber) are consistent with the prescription and are licensed

At 706, a dosage data generation operation is performed. In an embodiment, the system 102 is configured to generate dosage data for an infusion of a treatment fluid to the patient 122 based on the medication data and the patient data. The dosage data indicates a concentration associated with one or more medication fluids stored in the one or more medication containers of the plurality of medication containers for forming the treatment fluid. The dosage data may refer to a concentration, a quantity, and a timing required for delivering the treatment fluid based on the patient's needs and prescription data. For example, the system calculates that the required antibiotic dose is 1050 mg, which translates to 2.1 mL of antibiotic solution. The pain relief medication is added at a rate of 5 mL/hour, and the total infusion rate is determined.

In an embodiment, the system 102 generates patient-specific dosage data for an infusion of a treatment fluid to the patient based on the medication data and the prescription data associated. In an example, the system calculates that the blood thinner needs to be delivered at 1 mL/hour (from a 2 mg/mL solution), the chemotherapy medication requires delivery at 0.5 mL/hour (from a 2 mg/mL solution), and saline must flow at 10 mL/hour to maintain the proper flow. In another example, the system 102 calculates that the pain-relief medication needs to be delivered at 0.5 mL/hour (from a 10 mg/mL solution), the antibiotic at 0.5 mL/hour (from a 20 mg/mL solution), and saline at 15 mL/hour as the medication fluid.

At 708, a set of control parameters generation operation is performed. In an embodiment, the system 102 is configured to generate a set of control parameters associated with one or more pressurizing pumps of the plurality of pressurizing pumps 106, and one or more pressure valves of the plurality of pressure valves 112. The set of control parameters is generated based on the medication data and the dosage data. For example, the one or more pressurizing pumps 106 are associated with one or more medication containers of the plurality of medication containers 104A. In an embodiment the control parameters are used to manage the operation of various components in the system, such as but not limited to, pressurizing pumps 106 and pressure valves 112. The control parameters may determine the pressure levels, mixing ratios, and flow rates required for preparing and administering the treatment fluid. For example, the control parameters specify that pressurizing pump A dispenses saline at 20 mL/hour, pressurizing pump B delivers the antibiotic solution at 2.1 mL/hour, and the plurality of pressure valves helps in the mixing of medication fluids before entering the patient conduit 110.

In an example, the system 102 generates the set of control parameters to deliver the treatment fluid to the patient 122 based on the medication data and the patient-specific dosage data for the patient 122. The set of control parameters is associated with at least one of a set of medication containers of the plurality of medication containers 104A, a set of carrier conduits of the one or more carrier conduits 108, and the patient conduit 110. The set of control parameters is also associated with pressurizing pumps 106 associated with the set of medication containers and pressure valves 112 controlling flow across the set of carrier conduits, and the patient conduit 110. For example, the pressure valve 112 is controlled based on the set of control parameters to combine the blood thinner (1 mL/hour) and chemotherapy medication (0.5 mL/hour) into the saline stream (10 mL/hour) before administration.

At 710, a flow of the treatment fluid control operation is performed. In an embodiment, the system 102 is configured to control a flow of the treatment fluid based on the set of control parameters to deliver the treatment fluid to the patient. In an embodiment of this step, the treatment fluid is prepared and delivered accurately and safely to the patient in accordance with the prescribed dosage. For example, the system continuously monitors and adjusts the flow to maintain a consistent infusion rate of 27.1 mL/hour, blending the saline, antibiotic, and pain relief medication accurately. The pressurizing pumps 106 associated with the set of medication containers may be controlled to control the flow rates of medications and carrier fluids. For example, the pressurizing pump 404A is configured to dispense saline at 10 mL/hour, pressurizing pump 404B is set to deliver the blood thinner at 1 mL/hour, and pressurizing pump 404C is set to dispense the chemotherapy medication at 0.5 mL/hour.

In an embodiment, the delivery of medication to the patient 122 is continuously monitored to align with the calculated dosages and maintain accuracy. For example, the system provides a treatment fluid containing saline at 10 mL/hour, the blood thinner at 1 mL/hour, and the chemotherapy medication at 0.5 mL/hour to the patient 112A. Simultaneously, the system ensures that patient 122 is infused with saline at 15 mL/hour, the pain-relief medication at 0.5 mL/hour, and the antibiotic at 0.5 mL/hour.

In an example, the system 102 may facilitate controlling the flow of the treatment fluid for individualized patients and providing precise medication delivery for multiple patients by generating distinct sets of control parameters. The advanced design allows for independent operation, maintaining the flexibility and the safety in one or more patient environments. Further, the proposed system may mitigate wastage of the medication fluid by managing the integration and flow of treatment fluids to align with each of the one or more patients from the specific treatment plan.

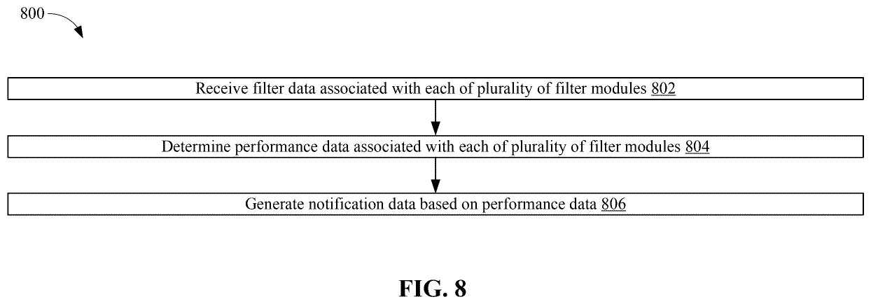

FIG. 8 illustrates a flowchart 800 of a method for generating notification data, in accordance with an embodiment of the disclosure. FIG. 8 is explained in conjunction with elements from FIG. 1A-1B, FIG. 2, FIG. 3, FIG. 4, FIG. 5, FIG. 6A-6B, and FIG. 7. With reference to FIG. 8 there is shown a flowchart 800. The operations of the exemplary method may be executed by any computing system, for example, by the system 102 of FIG. 1. The operations of the flowchart 800 may start at 802.

At 802, a filter data reception operation is performed. In an embodiment, the system 102 is configured to receive filter data associated with each of the plurality of filter modules 412, the filter data includes at least one of pressure change data, usage data, expiration data, anomaly data, or flow data. The filter data includes information such as but not limited to, the type, condition, usage duration, and filtration efficiency of each module. For example, filter module 114A may report a 90% filtration efficiency with a total usage time of 100 hours, while filter module 114B may report an efficiency of 70% with a usage time of 150 hours. The filter data may be received from sensors or monitoring devices integrated with each filter module 412, obtaining real-time updates on operational conditions.

At 804, a performance data determination operation is performed. In an embodiment, the system 102 is configured to determine performance data associated with each of the plurality of filter modules 412, the performance data indicates a current filtering capacity of each of the plurality of filter modules 412. The performance data may reflect the operational state and effectiveness of the filters, considering parameters such as but not limited to declining filtration efficiency, clogged material, or nearing replacement thresholds. For example, the system 102 may determine that filter module 412B has reached a critical threshold of efficiency decline (below 75%), suggesting an imminent need for replacement, while filter module 412A is operating optimally.

At 806, a notification data generation operation is performed. In an embodiment, the system 102 is configured to generate notifications data based on the performance data. The notification data is generated based on a determination of the current filtering capacity to be less than a capacity threshold. The notification data is formulated to inform relevant personnel or systems about the status of the filters and any required maintenance actions. Notifications may include alerts such as but not limited to, “Filter Module requires replacement due to reduced efficiency” or “Filter Module operating within acceptable parameters.” Such notifications are transmitted to healthcare staff, system operators, or maintenance personnel through user interfaces, mobile applications, or automated logs.

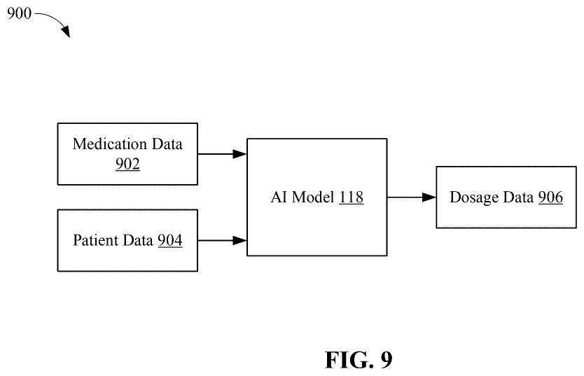

FIG. 9 illustrates a block diagram 900 to generate dosage data, in accordance with an embodiment of the disclosure. FIG. 9 is explained in conjunction with elements from FIG. 1, FIG. 1B, FIG. 2, FIG. 3, FIG. 4, FIG. 5, FIG. 6A-6B, FIG. 7, and FIG. 8.

In an embodiment, the system 102 receives medication data 902, as described, for example in FIG. 7. The medication data 902 includes details about the available medications, such as their type, concentration, volume, and storage conditions. For example, the system detects that one medication container stores a saline solution at 0.9% concentration, another container holds a blood thinner at 2 mg/mL, and a third container stores a chemotherapy medication at 5 mg/mL. The medication data 902 ensures that the system has accurate and up-to-date information on all available medications for precise dosing calculations.

Further, the system 102 receives patient data 904, as described, for example in FIG. 7. The patient data 904 provides comprehensive information about the patients requiring treatment. The patient data 904 includes patient-specific details such as but not limited to medical history, weight, age, current health conditions, and prescribed treatment plans. For example, the patient 122 data may indicate a requirement for 2 mg/hour of a blood thinner and 1 mg/hour of a chemotherapy medication, and the patient 122 requires 1.5 mg/hour of the same blood thinner and no chemotherapy medication. The patient may receive a dosage that is personalized corresponding to the medical needs of the patient. Using the received medication data 902 and the patient data 904, the AI model 118 generates the dosage data 906 tailored to the patient's treatment requirements. The AI model 118 analyses the inputs to calculate the exact volumes and flow rates of each medication to be delivered. For example, for the patient, the AI model 118 determines that the blood thinner should be administered at 1 mL/hour from a 2 mg/mL solution, while the chemotherapy medication should be delivered at 0.2 mL/hour from a 5 mg/mL solution. Similarly, for another patient, the blood thinner is calculated to flow at 0.75 mL/hour from the same 2 mg/mL solution.

Further, the AI model 118 may be applied on the medication data and the patient data comprising the prescription data. In one embodiment, the AI model is also applied to real-time patient vitals data. In such a case, the real-time patient vitals data may be obtained, for example, from multiple sensors or diagnosis equipment attached to the patient. The real-time patient vitals data may further include, but is not limited to height, weight, sex, age, temperature, blood pressure, heart rate, saturation, central venous saturation, heart rhythm, SED Line monitor information, EEG information, nerve stimulator information, neuro-monitoring information in real time. The real-time patient vitals data from the patient 122 will be utilized to alter the dosage data for the patient including allowed changes in the dosages in accordance with the prescriptions and/or prescriber real-time feedback.

In an embodiment, the integration of AI model 118 helps in high precision in dosage calculations, minimizing the risk of errors and enhancing the safety and efficacy of medication delivery. By leveraging real-time patient and medication data, the system maintains dynamic adjustments to dosage as required by changing patient conditions or treatment protocols. This advanced process supports healthcare providers in delivering tailored treatment fluids efficiently and effectively.

The system 102 may include a method to receive input concerning the patient's condition including but not limited to height, weight, sex, age, temperature, blood pressure, heart rate, saturation, heart rhythm, SED Line monitor information, EEG information, nerve stimulator information, neuro-monitoring information. It may integrate this information to make dose adjustment suggestions to the operator. The operator may put parameters into the system that would allow the system to automatically adjust dosages and/or infusion rates within those parameters based upon analysis of the input data in real-time and/or installed algorithms and/or installed and/or learned information from artificial intelligence modules.

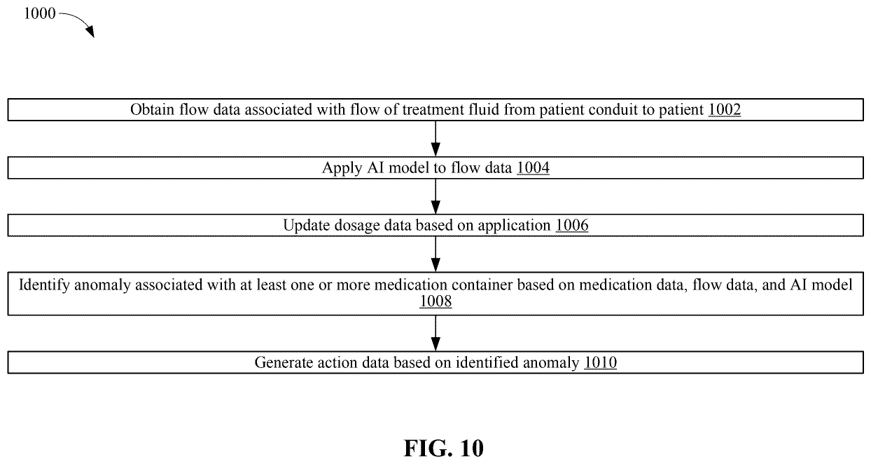

FIG. 10 illustrates a flowchart 1000 of a method for generating action data, in accordance with an embodiment of the disclosure. FIG. 10 is explained in conjunction with elements from FIG. 1A-1B, FIG. 2, FIG. 3, FIG. 4, FIG. 5, FIG. 6A-6B, FIG. 7, FIG. 8, and FIG. 9. The operations of the exemplary method may be executed by any computing system, for example, by the system 102 of FIG. 1. The operations of the flowchart 1000 may start at 1002.

At 1002, a flow data obtaining operation is performed. In an embodiment, the system 102 is configured to obtain flow data associated with a flow of the treatment fluid from the patient conduit 110 to the patient, the flow data is obtained from the dispensing pump. Flow data includes real-time measurements of parameters such as but not limited to, flow rate, pressure, and volume of the fluid being delivered to the patient. For example, the system measures that the saline solution flows at 10 mL/hour, the blood thinner flows at 1 mL/hour, and the chemotherapy medication flows at 0.5 mL/hour. This data provides insights into whether the treatment fluid is being administered as intended.

At 1004, an AI model 118 application operation is performed. In an embodiment, the system 102 is configured to apply the AI model 118 to the flow data. The AI model processes the flow data in combination with predefined treatment protocols and historical performance data to identify deviations or inefficiencies. For example, if the flow data indicates that the blood thinner is being dispensed at 0.8 mL/hour instead of the prescribed 1 mL/hour, the AI model detects this discrepancy and flags the discrepancy for further action.

At 1006, a dosage data update operation is performed. In an embodiment, the system 102 is configured to update the dosage data 906 based on the application of the AI model to the flow data. The updated dosage data 906 provides the treatment fluid delivery aligned with the prescribed protocol, considering any adjustments needed due to changes in patient condition or system performance. For example, if the AI model 118 identifies a need to increase the saline flow rate to compensate for reduced chemotherapy medication delivery, the system recalculates the required flow rates and updates the dosage data 906 accordingly.

At 1008, an anomaly identification operation is performed. In an embodiment, the system 102 is configured to identify an anomaly associated with at least one of the one or more medication containers based on the medication data, the flow data, and the AI model. Anomalies include issues such as but not limited to, low fluid levels, blockages, or incorrect dispensing rates from medication containers. For example, if the medication container for the blood thinner is nearly empty, causing a reduced flow rate, the system identifies this situation as an anomaly and categorizes it for resolution. In an embodiment, the anomaly may refer to any deviation, irregularity, or inefficiency identified by the system during the medication delivery process. The anomalies may include issues such as low fluid levels in a container, blockages in the dispensing mechanism, incorrect dispensing rates, or discrepancies between the prescribed and actual flow rates of medications. For instance, if the AI model 118 identifies that a medication container has an unexpected blockage reducing the flow rate, the system categorizes this as the anomaly. The detected anomaly serves as the trigger for generating action data, which may include adjusting system parameters to bypass the blockage or notifying healthcare staff to clear the blockage.

At 1010, an action data generation operation is performed. In an embodiment, the system 102 is configured to generate action data based on the identified anomaly. The action data includes at least one of one or more resolution parameters for resolving the anomaly, or notification data for a notification associated with the anomaly. The action data includes specific instructions to rectify the detected issue, such as alerting healthcare staff, adjusting pressure settings, or recommending container replacement. For example, if the chemotherapy medication container is found to have a blockage, the system generates a notification for healthcare staff to clear the blockage and suggests adjusting the pressurizing pump temporarily to maintain delivery. In an embodiment, the action data may refer to the specific information or instructions generated by the system in response to identifying an anomaly in the medication delivery process. The action data serves as the basis for resolving the anomaly or informing the relevant stakeholders. The action data includes one or more resolution parameters, such as adjustment values for system components (e.g., pressure settings, flow rates), or notification data, which consists of alerts sent to healthcare staff or system operators. For example, if the system detects that the blood thinner medication is being dispensed at a lower flow rate due to a nearly empty container, the action data may include resolution parameters such as increasing pressure to optimize the remaining medication flow or recommending container replacement and the action data may trigger a notification alerting healthcare personnel about the low fluid level, ensuring timely intervention.

In an embodiment, the process described in FIG. 10 provides a robust and adaptive treatment fluid delivery system. By integrating real-time data acquisition, AI analysis, and automated anomaly detection, the system minimizes errors, enhances safety, and ensures that patients receive treatment fluids.

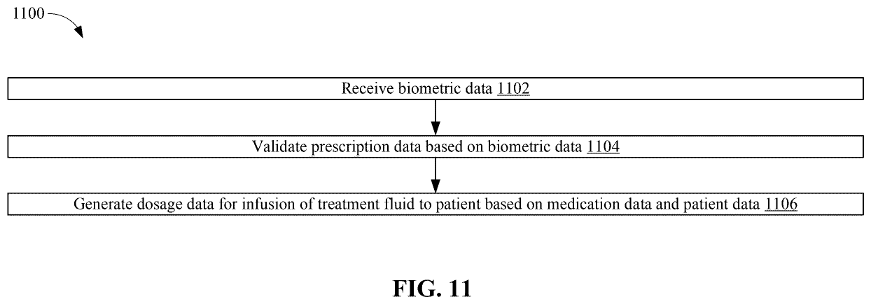

FIG. 11 illustrates a flowchart 1100 of a method for generating dosage data, in accordance with an embodiment of the disclosure. FIG. 11 is explained in conjunction with elements from FIG. 1A-1B, FIG. 2, FIG. 3, FIG. 4, FIG. 5, FIG. 6A-6B, FIG. 7, FIG. 8, FIG. 9, and FIG. 10. The operations of the exemplary method may be executed by any computing system, for example, by the system 102 of FIG. 1. The operations of the flowchart 1100 may start at 1102.

At 1102, a biometric data reception operation is performed. In an embodiment, the system 102 is configured to receive the biometric data. The biometric data is associated with a patient 122. The biometric data may include parameters such as heart rate, blood pressure, and oxygen saturation, wherein the biometric data parameters are essential in determining the correct dosage for the patient 122. For example, the patient 122 with low blood pressure may require a lower dosage of medication to avoid any adverse effects or a dosage, or increased dosage of medication to raise the blood pressure. In an example, the biometric data may further include information associated with the healthcare professional responsible for writing the prescription or administering the medication.

At 1104, a prescription data validation operation is performed. In an embodiment, the system 102 is configured to validate the prescription data based on the biometric data. The validation of prescription data ensures that the prescribed medication or treatment aligns with the patient's current health status. In an embodiment, if discrepancies are found, such as the prescribed medication could potentially harm the patient 122 based on biometric data such as an allergy to the medication, then the system 102 may trigger alerts or suggest modifications in the medication delivery to the patient 112.

In an example, the system 102 is configured to validate rights of the healthcare professional to use the system 102 based on the biometric data. These rights may include rights to create, modify or follow the prescription data.

At 1106, a dosage data generation operation is performed. In an embodiment, the system 102 is configured to generate the dosage data 906 for the infusion of the treatment fluid to the patient based on the validation. This ensures that the dosage is appropriate for the patient's condition and medical history. For example, the patient 122 has diabetes so the dosage of insulin for the patient 122 is adjusted according to real-time blood glucose readings.

FIG. 12 illustrates a flowchart of an exemplary method for controlling medical fluid infusion for the patients, in accordance with an embodiment of the present disclosure. FIG. 12 is explained in conjunction with FIG. 1A, FIG. 1B, FIG. 2, FIG. 3., FIG. 4, FIG, 5, FIG. 6A-6B, FIG. 7, FIG. 8, FIG. 9, FIG. 10, and FIG. 11. The system 102 may include at least one processor (hereinafter, referred to as “processor 1202”), at least one memory (hereinafter, referred to as “memory 1204”), I/O interface 1206, and communication interface 1208. The processor 1202 may be connected to the memory 1204, the I/O interface 1206, and the communication interface 1208 through one or more wired or wireless connections. Although in FIG. 12, it is shown that the system 102 includes the processor 1202, the memory 1204, the I/O interface 1206, and the communication interface 1208, however, the disclosure may not be so limiting and the system 102 may include fewer or more components to perform the same or other functions of the system 102.

The processor 1202 may be configured to control medical fluid infusion for the patients. The processor 1202 may be embodied in several different ways. For example, the processor 1202 may be embodied as one or more of various hardware processing means such as a coprocessor, a microprocessor, a controller, a digital signal processor (DSP), a processing element with or without an accompanying DSP, or various other processing circuitry including integrated circuits such as an ASIC (application specific integrated circuit), an FPGA (field programmable gate array), a microcontroller unit (MCU), a hardware accelerator, a special-purpose computer chip, or the like. As such, in some embodiments, the processor 1202 may include one or more processing cores configured to perform independently.

For example, when the processor 1202 may be embodied as an executor of computer program code instructions, the instructions may specifically configure the processor 1202 to perform the algorithms and/or operations described herein when the instructions are executed. However, in some cases, the processor 1202 may be a processor-specific device (for example, a mobile terminal or a fixed computing device) configured to employ an embodiment of the present disclosure by further configuration of the processor 1202 by instructions for performing the algorithms and/or operations described herein. The processor 1202 may include, among other things, a clock, an arithmetic logic unit (ALU), and logic gates configured to support the operation of the processor 1202.

The memory 1204 may be non-transitory and may include, for example, one or more volatile and/or non-volatile memories. In other words, for example, the memory 1204 may be an electronic storage device (for example, a computer readable storage medium) including gates configured to store data (for example, bits) that may be retrievable by a machine (for example, a computing device like the processor 1202). The memory 1204 may be configured to store information, data, content, applications, instructions, or the like, for enabling the system 102 to carry out various functions in accordance with an example embodiment of the present disclosure. For example, the memory 1204 may be configured to buffer input data for processing by the processor 1202. As exemplarily illustrated in FIG. 12, the memory 1204 may be configured to store instructions for execution by the processor 1202. As such, whether configured by hardware or software methods, or by a combination thereof, the processor 1202 may represent an entity (for example, physically embodied in circuitry) capable of performing operations according to an embodiment of the present disclosure while configured accordingly. Thus, for example, when the processor 1202 is embodied as an Application Specific Integrated Circuit (ASIC), Field Programmable Gate Array (FPGA), or the like, the processor 1202 may be specifically configured hardware for conducting the operations described herein. In an embodiment, the memory 1204 may be configured to store an AI Model 118, medication data 902, patient data 904, dosage data 906, and a set of control parameters 1204A.

In some example embodiments, the I/O interface 1206 may be configured to receive the input and/or output generated by the system 102. In an embodiment, the I/O interface 1206 may be configured to communicate with the system 102 and display the input and/or output of the system 102. As such, the I/O interface 1206 may include a display screen and, in some embodiments, may also include a keyboard, a mouse, a joystick, a touch screen, touch areas, soft keys, one or more microphones, a plurality of speakers, or other input/output mechanisms. In one embodiment, the system 102 may include a user interface circuitry configured to control at least some functions of one or more I/O interface elements such as a display device and, in some embodiments, a plurality of speakers, a ringer, one or more microphones and/or the like. In an embodiment, the I/O interface 1206 may include an input interface and output interface for supporting communications to and from the system 102 or any other component with which the system 102 may communicate. The processor 1202 may be configured to control one or more functions of one or more I/O interface 1206 elements through computer program instructions (for example, software and/or firmware) stored on the memory 1204 accessible to the processor 1202.

The communication interface 1208 may be any means such as a device or circuitry embodied in either hardware or a combination of hardware and software that is configured to receive and/or transmit data to/from other communication devices in communication with the system 102. In this regard, the communication interface 1208 may include, for example, one or more antennas and supporting hardware and/or software for enabling communications with a wireless communication network. Additionally, or alternatively, the communication interface 1208 may include the circuitry for interacting with the antenna(s) to cause transmission of signals via the antenna(s) or to handle receipt of signals received via the antenna(s). In some environments, the communication interface 1208 may alternatively or additionally support wired communication. As such, for example, the communication interface 1208 may include a communication port, a communication modem, and/or other hardware and/or software for supporting communication via cable, digital subscriber line (DSL), universal serial bus (USB), or other mechanisms. The system 102 may contain a method to integrate with ‘hospitals’, ‘surgery centers’, ‘clinics’, or ‘other healthcare systems’ electronic medical record systems (“EMR's”) via WIFI, Bluetooth®, radiofrequency or other means.

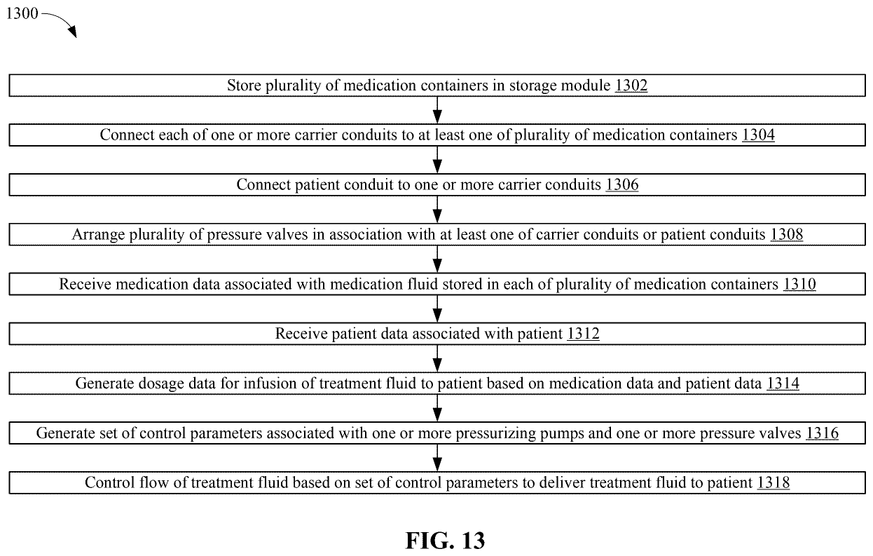

FIG. 13 illustrates a flowchart of an exemplary method for controlling medical fluid infusion for the patients, in accordance with an embodiment of the disclosure. FIG. 13 is explained in conjunction with elements from FIG. 1A-1B, FIG. 2, FIG. 3, FIG. 4, FIG. 5, FIG. 6A-6B, FIG. 7, FIG. 8, FIG. 9, FIG. 10, FIG. 11, and FIG. 12. With reference to FIG. 13 there is shown a flowchart 1300. The operations of the exemplary method may be executed by any computing system, for example, by the system 102 of FIG. 1 or the processor 1202 of FIG. 12. The operations of the flowchart 1300 may start at 1302.

At 1302, a plurality of medication containers may be stored in a storage module. In an embodiment, the plurality of medication containers 104A are stored in the storage module 104. Data related to the medication containers 104A is accessed by the processor 1202 and stored in memory 1204. Each of the plurality of medication containers 104A stores a medication fluid. Details associated with the storage of the plurality of medication containers 104A are provided, for example in FIG. 1A-1B, FIG. 2 and FIG. 3.

At 1304, each of one or more carrier conduits 108 may be connected to at least one of the plurality of medication containers 104A. In an embodiment, the processor 1202 may connect information about and control of each of the one or more carrier conduits 108 to at least one of the plurality of medication containers 104A. Details associated with the connection of the one or more carrier conduits 108 are provided for example, in FIG. 4.

At 1306, a patient conduit 110 may be connected to the one or more carrier conduits 108. In an embodiment, the processor 1202 may connect flow to the patient conduit 110 from one or more carrier conduits 108. The patient conduit 110 may be configurable to connect to the patient 122. Details associated with the connection of the patient conduit 110 are provided for example, in FIG. 4.

At 1308, a plurality of pressure valves 112 may be arranged in association with at least one of the one or more carrier conduits 108, or the patient conduit 110. In an embodiment, the processor 1202 may control the plurality of pressure valves 112 in association with at least one of the one or more carrier conduits 108, or the patient conduit 110. Details associated with the arrangement of the pressure valves 112 are provided for example in FIG. 4.

At 1310, medication data 902 associated with the medication fluid may be received. In an embodiment, the processor 1202 may receive the medication data 902 associated with medication fluid stored in each of the plurality of medication containers 104A. Details associated with the reception of the medication data 902 are provided, for example, in FIG. 7.

At 1312, patient data 904 may be received. In an embodiment, the processor 1202 may receive the patient data 904 associated with the patient 122. The patient data 904 includes prescription data. Details associated with the reception of the patient data 904 are provided, for example, in FIG. 7.

At 1314 dosage data 906 may be generated. In an embodiment, the processor 1202 may generate dosage data 906 for an infusion of a treatment fluid to the patient based on the medication data 902 and the patient data 904. The dosage data 906 indicates a concentration associated with one or more medication fluids stored in the one or more medication containers of the plurality of medication containers 104A for forming the treatment fluid. Details associated with the generation of the dosage data 906 are provided for example, in FIG. 7.

At 1316, a set of control parameters may be generated. In an embodiment, the processor 1202 may generate a set of control parameters associated with one or more pressurizing pumps 106 of the plurality of pressurizing pumps 106, and one or more pressure valves 112 of the plurality of pressure valves 112. The set of control parameters is generated based on the medication data 902 and the patient data 904, and the one or more pressurizing pumps 106 are associated with the one or more medication containers 104A. Details associated with the generation of the set of control parameters are provided for example, in FIG. 7.

At 1318, a flow of the treatment fluid may be controlled. In an embodiment, the processor 1202 may control a flow of the treatment fluid based on the set of control parameters to deliver the treatment fluid to the patient. Details associated with the controlling of the flow of the treatment fluid are provided, for example, in FIG. 7.

Alternatively, the system 102 may include means for performing each of the operations described above. In this regard, according to an example embodiment, examples of means for performing operations may include, for example, the processor and/or a device or circuit for executing instructions or executing an algorithm for processing information as described above.

Various embodiments of the disclosure may provide a non-transitory computer-readable medium having stored thereon computer executable instructions, which when executed by one or more processors (such as the processor 1202), cause the one or more processors 1202 to carry out operations to operate a system (e.g., the system 102) for controlling medical fluid infusion for patients. The instructions may cause the machine and/or computer to perform operations including receiving medication data 902 associated with the medication fluid stored in each of a plurality of medication containers 104A. The plurality of medication containers is stored in a storage module 104, and each of the plurality of medication containers 104A stores a medication fluid. The operation further includes receiving patient data 904 associated with a patient 122. The patient data 904 includes prescription data. The operation further includes applying an artificial intelligence (AI) model 118 to the medication data 902 and the prescription data. The operation further includes generating dosage data 906 for an infusion of a treatment fluid to the patient based on the application of the AI model 118 to the medication data 902 and the prescription data. The dosage data 906 indicates a concentration associated with one or more medication fluids stored in the one or more medication containers 104A of the plurality of medication containers 104A for forming the treatment fluid. The operation further includes generating a set of control parameters associated with one or more pressurizing pumps 106 of a plurality of pressurizing pumps 106, and one or more pressure valves 112 of a plurality of pressure valves 112. The set of control parameters is generated based on the medication data 902 and the dosage data 906, and the one or more pressurizing pumps 106 are associated with the one or more medication containers 104A. The operation further includes controlling a flow of the treatment fluid based on the set of control parameters to deliver the treatment fluid to the patient 122.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of reactants and/or functions, it should be appreciated that different combinations of reactants and/or functions may be provided by alternative embodiments without departing from the scope of the appended claims. In this regard, for example, different combinations of reactants and/or functions than those explicitly described above are also contemplated as may be set forth in some of the appended claims. Although specific terms are employed herein, they are used in a generic and descriptive sense only and not for purposes of limitation.

What is claimed is:

1. A system for controlling medical fluid infusion for patients, the system comprising:
a storage module for storing a plurality of medication containers, wherein each of the plurality of medication containers stores a medication fluid;
a plurality of pressurizing pumps, wherein
each of the plurality of pressurizing pumps is arranged in connection with a corresponding medication container of plurality of medication containers, and
each of the plurality of pressurizing pumps is configurable to apply a pressure to the corresponding medication container of the plurality of medication containers;
one or more carrier conduits, wherein each of the one or more carrier conduits is connected to at least one of the plurality of medication containers;
a patient conduit connected to the one or more carrier conduits, wherein the patient conduit is configurable to connect to a patient;
a plurality of pressure valves arranged in association with at least one of: the one or more carrier conduits, or the patient conduit;
a memory configured to store computer-executable instructions; and
one or more processors coupled to the storage module, the plurality of pressurizing pumps, and the plurality of pressure valves, wherein the one or more processors are configured to execute the computer-executable instructions to:
receive medication data associated with the medication fluid stored in each of the plurality of medication containers;
receive patient data associated with the patient, wherein the patient data comprises prescription data;
generate dosage data for an infusion of a treatment fluid to the patient based on the medication data and the patient data, wherein the dosage data indicates a concentration associated with one or more medication fluids stored in the one or more medication containers of the plurality of medication containers for forming the treatment fluid;
generate a set of control parameters associated with one or more pressurizing pumps of the plurality of pressurizing pumps, and one or more pressure valves of the plurality of pressure valves, wherein the set of control parameters is generated based on the medication data and the dosage data, and wherein the one or more pressurizing pumps are associated with the one or more medication containers; and
control a flow of the treatment fluid based on the set of control parameters to deliver the treatment fluid to the patient.

2. The system of claim 1, wherein at least one of the plurality of medication containers corresponds to a multi-chamber container, and wherein the multi-chamber container comprises:
a first chamber configured to store a first medication entity, and wherein the first medication entity corresponds to at least one of: a powdered state, a solid state, or a semi-solid state;
a second chamber configured to store a second medication entity, wherein the second medication entity corresponds to a liquid state; and
a mixing mechanism associated with the multi-chamber container, wherein the mixing mechanism is coupled to the one or more processors.

3. The system of claim 2, wherein the one or more processors are further configured to:
identify the multi-chamber container from the plurality of medication containers based on the dosage data, wherein the first medication entity and the second medication entity of the multi-chamber container are combined to form the treatment fluid;
generate the set of control parameters based on the identification; and
control the mixing mechanism to combine the first medication entity and the second medication entity to form the treatment fluid based on at least a part of the set of control parameters.

4. The system of claim 1, wherein the second medication entity is a pressurized fluid, and wherein the pressurized fluid corresponds to one of: a gas, a liquid, a Newtonian fluid, a non-Newtonian fluid to increase a pressure in the multi-chamber container.

5. The system of claim 1, wherein the plurality of pressurizing pumps corresponds to compressed gas pumps, and wherein each of the plurality of pressurizing pumps is configured to apply the pressure for the corresponding medication container of the plurality of medication containers using a compressed gas.

6. The system of claim 1, wherein the plurality of pressurizing pumps corresponds to peristaltic pumps, and wherein each of the plurality of pressurizing pumps is configured to apply the pressure for the corresponding medication container of the plurality of medication containers using a compressible flexible tubing.

7. The system of claim 1, wherein the plurality of pressurizing pumps corresponds to hydraulic pumps, and wherein each of the plurality of pressurizing pumps is configured to apply the pressure for the corresponding medication container of the plurality of medication containers using a liquid medium.

8. The system of claim 1, wherein the treatment fluid comprises a medication fluid stored within a medication container of the plurality of medication containers or a combination of a set of medication fluids stored within the one or more medication containers of the plurality of medication containers.

9. The system of claim 1, wherein the one or more processors are further configured to:
identify the one or more medication containers from the plurality of medication containers based on the dosage data, wherein the medication fluid from each of the one or more medication containers is combined to form the treatment fluid;
identify the one or more pressure valves from the plurality of pressure valves, wherein the one or more pressure valves are associated with at least one of: a connection between the identified one or more medication containers and at least one carrier conduit of the one or more carrier conduits, the at least one carrier conduit, or the patient conduit; and
generate the set of control parameters for controlling each of the one or more pressurizing pumps and the one or more pressure valves, wherein the one or more pressurizing pumps are associated with the identified one or more medication containers, and wherein the set of control parameters indicates a pressure value and a flow rate.

10. The system of claim 9, wherein the one or more processors are further configured to:
determine pressure data based on the dosage data, wherein the pressure data comprises a first pressure value associated with the one or more pressurizing pumps, a second pressure value associated with the connection between each of the one or more medication containers and the at least one carrier conduit, a third pressure value associated with the at least one carrier conduit, and a fourth pressure value associated with the patient conduit; and
generate the set of control parameters for controlling each of the one or more pressurizing pumps and the one or more pressure valves, based on the pressure data.

11. The system of claim 10, wherein
the second pressure value is lesser than the first pressure value, the third pressure value is lesser than the second pressure value, and the fourth pressure value is lesser than the third pressure value, and
the control of each of the one or more pressurizing pumps and the one or more pressure valves is based on the corresponding set of control parameters, such that the pressure data prevents backflow of the treatment fluid and contamination of any one of: the at least one carrier conduit, the patient conduit, or the plurality of medication containers.

12. The system of claim 1, wherein a new disposable patient conduit is placed and configurable to connect to a subsequent patient after the delivery of the treatment fluid to the patient, and wherein the one or more processors are configured to cause to deliver the treatment fluid or new treatment fluid to the subsequent patient.

13. The system of claim 1, wherein a first label is positioned on each of the plurality of medication containers, and wherein the system further comprises a scanner, wherein
the scanner is configured to read the first label of each of the plurality of medication containers, and
the one or more processors are configured to retrieve the medication data associated with each of the plurality of medication containers based on the reading.

14. The system of claim 1, further comprising a plurality of filter modules, wherein
each of the plurality of filter modules is arranged in connection with at least one of: the one or more carrier conduits, or the patient conduit,
each of the plurality of filter modules comprises at least one microbe filter to filter one or more microbe particles, and
each of the plurality of filter modules comprises at least one blood cell filter to filter blood-particulate matter.

15. The system of claim 14, wherein the processor is further configured to:
receive filter data associated with each of the plurality of filter modules, wherein the filter data comprises at least one of: pressure change data, usage data, expiration data, anomaly data, or flow data;
determine performance data associated with each of the plurality of filter modules, wherein the performance data indicates a current filtering capacity of each of the plurality of filter modules; and
generate notifications data based on the performance data, wherein the notification data is generated based on a determination of the current filtering capacity to be less than a capacity threshold.

16. The system of claim 14, wherein each of the at least one microbe filter and the at least one blood cell filter are selected based on a pore size.

17. The system of claim 14, wherein a second label is positioned on each filter module of the plurality of filter modules, and wherein the system further comprises a scanner, wherein
the scanner is configured to read the second label of each of the plurality of filter modules; and
the one or more processors are configured to retrieve the filter data associated with each of the plurality of filter modules based on the reading.

18. The system of claim 14, further comprising a sterilization module arranged in connection with each of the plurality of filter modules, wherein the sterilization module is configured to periodically sterilize each of the plurality of filter modules.

19. The system of claim 18, the sterilization module is configured to sterilize each of the one or more filter modules using at least one of: an ultraviolet (UV) light, or chemical agent.

20. The system of claim 1, wherein the one or more processors are further configured to:
apply an artificial intelligence (AI) model to the medication data and the patient data; and
generate the dosage data based on the application of the AI model to the medication data and the patient data, wherein the dosage data indicates one or more delivery parameters to deliver the treatment fluid to the patient.

21. The system of claim 20, wherein the one or more processors are further configured to:
obtain real-time patient vitals data associated with the patient, wherein the patient vitals data comprises at least one of: height, weight, sex, age, temperature, blood pressure, heart rate, saturation, central venous saturation, heart rhythm, erythrocyte sedimentation rate (SED) Line monitor information, electroencephalogram (EEG) information, nerve stimulator information, or neuro-monitoring information;
apply the artificial intelligence (AI) model to the real-time patient vitals data; and
generate the dosage data based on the application of the AI model to the real-time patient vitals data.

22. The system of claim 20, wherein the system further comprises a dispensing pump to deliver the treatment fluid from the patient conduit to the patient, and wherein the one or more processors are further configured to:
obtain flow data associated with a flow of the treatment fluid from the patient conduit to the patient, wherein the flow data is obtained from the dispensing pump;
apply the AI model to the flow data; and
update the dosage data based on the application of the AI model to the flow data.

23. The system of claim 20, wherein the one or more delivery parameters indicate at least one of: the one or more medication fluids for forming the treatment fluid, a mixing ratio for forming the treatment fluid, a time period for the delivery of the treatment fluid to the patient, a concentration of the treatment fluid, or a flow rate of the treatment fluid.

24. The system of claim 20, wherein the one or more processors are further configured to:
identify an anomaly associated with at least one of the one or more medication containers based on the medication data, the flow data, and the AI model; and
generate action data based on the identified anomaly, wherein the action data comprises at least one of: one or more resolution parameters for resolving the anomaly, or notification data for a notification associated with the anomaly.

25. The system of claim 1, wherein the one or more processors are further configured to:
receive the biometric data, wherein the biometric data is associated with a healthcare professional;
validate the prescription data based on the biometric data; and
generate the dosage data for the infusion of the treatment fluid to the patient based on the validation.

26. The system of claim 1, wherein the plurality of medication containers comprises one or more sets of medication containers, such that one or more medication containers in a set of medication containers are connected in a series configuration with one of the one or more carrier conduits, and each set of the one or more sets of medication containers is connected in a parallel configuration with the one or more carrier conduits.

27. A method for controlling medical fluid infusion for patients, the method comprising:
storing a plurality of medication containers in a storage module, wherein each of the plurality of medication containers stores a medication fluid;
connecting each of one or more carrier conduits to at least one of the plurality of medication containers;
connecting a patient conduit to the one or more carrier conduits, wherein the patient conduit is configurable to connect to a patient;
arranging a plurality of pressure valves in association with at least one of: the one or more carrier conduits, or the patient conduit;
receiving medication data associated with the medication fluid stored in each of the plurality of medication containers;
receiving patient data associated with the patient, wherein the patient data comprises prescription data;
generating dosage data for an infusion of a treatment fluid to the patient based on the medication data and the patient data, wherein the dosage data indicates a concentration associated with one or more medication fluids stored in the one or more medication containers of the plurality of medication containers for forming the treatment fluid;
generating a set of control parameters associated with one or more pressurizing pumps of a plurality of pressurizing pumps, and one or more pressure valves of the plurality of pressure valves, wherein the set of control parameters is generated based on the medication data and the patient data, and wherein the one or more pressurizing pumps are associated with the one or more medication containers; and
controlling a flow of the treatment fluid based on the set of control parameters to deliver the treatment fluid to the patient.

28. The method of claim 27, further comprising:
identifying a multi-chamber container from the plurality of medication containers based on the dosage data, wherein the multi-chamber container comprises a first chamber configured to store a first medication entity, a second chamber configured to store a second medication entity, and a mixing mechanism associated with the multi-chamber container, and wherein the first medication entity and the second medication entity of the multi-chamber container are combined to form the treatment fluid;
generating the set of control parameters based on the identification; and
controlling a mixing mechanism to combine the first medication entity and the second medication entity to form the treatment fluid based on at least a part of the set of control parameters.

29. The method of claim 27, further comprising:
identifying the one or more medication containers from the plurality of medication containers based on the dosage data, wherein the medication fluid from each of the one or more medication containers is combined to form the treatment fluid;
identifying the one or more pressure valves from the plurality of pressure valves, wherein the one or more pressure valves are associated with at least one of: a connection between the identified one or more medication containers and at least one carrier conduit of the one or more carrier conduits, the at least one carrier conduit, or the patient conduit; and
generating the set of control parameters for controlling each of the one or more pressurizing pumps and the one or more pressure valves, wherein the one or more pressurizing pumps are associated with the identified one or more medication containers, and wherein the set of control parameters indicates a pressure value and a flow rate.

30. The method of claim 29, further comprising:
determining pressure data based on the dosage data, wherein the pressure data comprises a first pressure value associated with the one or more pressurizing pumps, a second pressure value associated with the connection between each of the one or more medication containers and the at least one carrier conduit, a third pressure value associated with the at least one carrier conduit, and a fourth pressure value associated with the patient conduit; and
generating the set of control parameters for controlling each of the one or more pressurizing pumps and the one or more pressure valves, based on the pressure data, wherein the second pressure value is lesser than the first pressure value, the third pressure value is lesser than the second pressure value, and the fourth pressure value is lesser than the third pressure value.

31. The method of claim 27, further comprising:
applying an artificial intelligence (AI) model to the medication data and the patient data; and
generating the dosage data based on the application of the AI model to the medication data and the patient data, and
wherein the dosage data indicates one or more delivery parameters to deliver the treatment fluid to the patient.

32. The method of claim 31, further comprising:
obtaining real-time patient vitals data associated with the patient, wherein the patient vitals data comprises at least one of: height, weight, sex, age, temperature, blood pressure, heart rate, saturation, central venous saturation, heart rhythm, erythrocyte sedimentation rate (SED) Line monitor information, electroencephalogram (EEG) information, nerve stimulator information, or neuro-monitoring information;
applying the artificial intelligence (AI) model to the real-time patient vitals data; and
generating the dosage data based on the application of the AI model to the real-time patient vitals data.

33. The method of claim 27, further comprising:
receiving filter data associated with each of a plurality of filter modules, wherein
each of the plurality of filter modules is arranged in connection with at least one of: the one or more carrier conduits, or the patient conduit,
each of the plurality of filter modules comprises at least one microbe filter to filter one or more microbe particles and at least one blood cell filter to filter blood-particulate matter, and
the filter data comprises at least one of: pressure change data, usage data, expiration data, anomaly data, or flow data;
determining performance data associated with each of the plurality of filter modules, wherein the performance data indicates a current filtering capacity associated with each of the plurality of filter modules associated with each of the plurality of filter modules; and
generating notifications data based on the performance data, wherein the notification data is generated based on a determination of the current filtering capacity to be less than a capacity threshold.

34. A computer programmable product comprising a non-transitory computer-readable medium having stored thereon computer-executable instructions, which when executed by one or more processors, cause the one or more processors to conduct operations, the operations comprising:
receiving medication data associated with a medication fluid stored in each of a plurality of medication containers, wherein the plurality of medication containers is stored in a storage module, and wherein each of the plurality of medication containers stores a medication fluid;
receiving patient data associated with a patient, wherein the patient data comprises prescription data;
applying an artificial intelligence (AI) model to the medication data and the patient data;
generating dosage data for an infusion of a treatment fluid to the patient based on the application of the AI model to the medication data and the patient data, wherein the dosage data indicates a concentration associated with one or more medication fluids stored in the one or more medication containers of the plurality of medication containers for forming the treatment fluid;
generating a set of control parameters associated with one or more pressurizing pumps of a plurality of pressurizing pumps, and one or more pressure valves of a plurality of pressure valves, wherein the set of control parameters is generated based on the medication data and the dosage data, and wherein the one or more pressurizing pumps are associated with the one or more medication containers; and
controlling a flow of the treatment fluid based on the set of control parameters to deliver the treatment fluid to the patient.
